(19) United States
(12) Patent Application Publication
(10) Pub. No.: US 2012/0065181 A1
(43) Pub. Date: Mar. 15, 2012

(54) METHOD OF PROVIDING NEUROPROTECTION USING SUBSTITUTED PORPHYRINS

(76) Inventors: David S. Warner, Chapel Hill, NC (US); Ines Batinic-Haberle, Durham, NC (US); Huaxin Sheng, Chapel Hill, NC (US); Ivan Spasojevic, Durham, NC (US)

(21) Appl. No.: 13/322,072

(22) PCT Filed: May 26, 2010
(86) PCT No.: PCT/US10/36256

§ 371 Date: Nov. 22, 2011

Related U.S. Application Data
(60) Provisional application No. 61/224,606, filed on Jul. 10, 2009.
(60) Provisional application No. 61/181,273, filed on May 26, 2009.

(51) Int. Cl. A61K 31/555; A61P 25/00; A61P 7/04; A61P 9/10

(52) U.S. Cl. 514/185

(57) ABSTRACT

Described herein are methods of treating ischemic injury comprising administering to a subject in need thereof a therapeutically effective amount of a substituted porphyrin compound. Also disclosed are methods of providing neuroprotection, methods of treating subarachnoid hemorrhage, methods of treating traumatic brain injury and methods of treating spinal cord injury using substituted porphyrins.

### CROSS-REFERENCE TO RELATED APPLICATIONS

This application claims priority under 35 U.S.C. §119(e) to U.S. Provisional Patent Application No. 61/181,273, filed May 26, 2009, and U.S. Provisional Patent Application No. 61/224,606, filed Jul. 10, 2009, each of which is incorporated by reference in its entirety.

### STATEMENT OF GOVERNMENT SUPPORT

This invention was made with U.S. Government support awarded by National Institutes of Health, Grant No. P01HL42444. The United States has certain rights in this invention.

### BACKGROUND

Sustained oxidative stress is a sequel to cerebral ischemia. A pro-oxidative state can induce direct tissue damage and also participates in regulation of the brain's delayed response to injury. Antioxidants have been demonstrated to ameliorate ischemic brain injury. However, most preclinical trials have utilized post-ischemic observation intervals of several hours to days to define antioxidant efficiency.

Post-ischemic histologic and neurologic responses to ischemia persist for weeks after perfusion has been restored. This is relevant to translation of preclinical studies to clinical trials, which typically assess outcome at intervals of several months post-ictus. Therefore, observations made in the first few days after experimental stroke may not predict efficacy in long-term outcome clinical trials.

### SUMMARY OF THE INVENTION

In one aspect, the present invention may provide a method of treating ischemic injury comprising administering a therapeutically effective amount of a compound of formula (I):

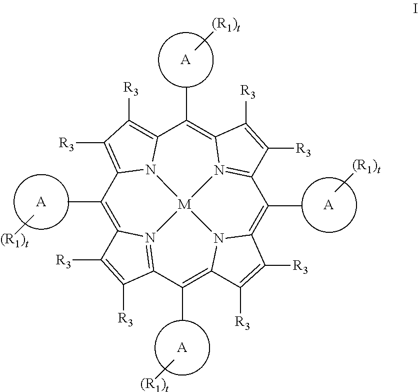

wherein:

each A is independently a heteroaryl group;

each R₁ is independently selected from H, C₆₋₁₂ alkyl, —(CH₂)ₙOR₂, —(CH₂)ₙSR₂, —(CH₂)ₙNR₂R₂, —(CH₂)ₙC(O)OR₄ and —(CH₂)ₘCHₚX_{q};

each R₂ is independently selected from hydrogen, alkyl, haloalkyl and —C(O)R₄;

each R₃ is independently selected from hydrogen, halogen, hydroxyl, alkoxy, alkyl, alkenyl, alkynyl, cycloalkyl, heterocycloalkyl, amino, amide, nitro, carboxylic acid, carboxyl, aryl, heteroaryl, thiol, thioalkyl, thioester, disulfide, phosphine, carbonyl, carbonylamino, formyl, sulfonyl, sulfonylamino, cyano, isocyano, C₁₋₄ alkyl aryl and C₁₋₄ alkyl heteroaryl;

each R₄ is independently selected from hydrogen, halogen, hydroxyl, alkoxy, alkyl, alkenyl, alkynyl, cycloalkyl, heterocycloalkyl, amino, amide, nitro, carboxylic acid, carboxyl, aryl, heteroaryl, thiol, thioalkyl, thioester, disulfide, phosphine, carbonyl, carbonylamino, formyl, sulfonyl, sulfonylamino, cyano, isocyano, C₁₋₄ alkyl aryl and C₁₋₄ alkyl heteroaryl;

each X is independently a halogen;

n is 1 to 12;

m is 1 to 11;

p is 0 to 3;

q is 0 to 3;

t is 0 to 2;

wherein p+q is 3;

M is selected from Mn, Fe, Co, Ni, Cu, V or 2 hydrogens; and wherein when said compound bears a charge, the compound further comprises one or more counterions;

to a subject in need thereof more than 4.5 hours post ischemia onset.

In another aspect, the present invention may provide a method of treating ischemic injury comprising administering a therapeutically effective amount of a compound of formula (I):

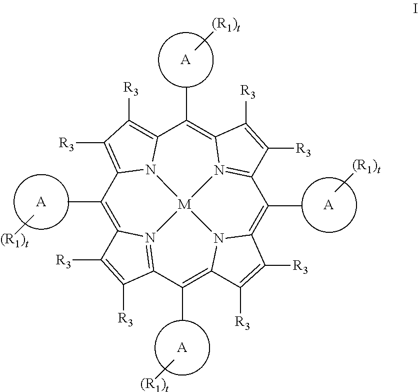

wherein:

each A is independently a heteroaryl group;

each R₁ is independently selected from H, C₆₋₁₂ alkyl, —(CH₂)ₙ0R₂, —(CH₂)ₙSR₂, —(CH₂)ₙNR₂R₂, —(CH₂)ₙC(O)OR₄ and —(CH₂)ₘCHₚX_{q};

each R₂ is independently selected from hydrogen, alkyl, haloalkyl and —C(O)R₄;

each R₃ is independently selected from hydrogen, halogen, hydroxyl, alkoxy, alkyl, alkenyl, alkynyl, cycloalkyl, heterocycloalkyl, amino, amide, nitro, carboxylic acid, carboxyl, aryl, heteroaryl, thiol, thioalkyl, thioester, disulfide, phosphine, carbonyl, carbonylamino, formyl, sulfonyl, sulfonylamino, cyano, isocyano, C₁₋₄ alkyl aryl and C₁₋₄ alkyl heteroaryl;

each R₄ is independently selected from hydrogen, halogen, hydroxyl, alkoxy, alkyl, alkenyl, alkynyl, cycloalkyl, heterocycloalkyl, amino, amide, nitro, carboxylic acid, carboxyl, aryl, heteroaryl, thiol, thioalkyl, thioester, disulfide, phosphine, carbonyl, carbonylamino, formyl, sulfonyl, sulfonylamino, cyano, isocyano, C₁₋₄ alkyl aryl and C₁₋₄ alkyl heteroaryl;

each X is independently a halogen;

n is 1 to 12;

m is 1 to 11;

p is 0 to 3;

q is 0 to 3;

t is 0 to 2;

wherein p+q is 3; and

M is selected from Mn, Fe, Co, Ni, Cu, V or 2 hydrogens;

wherein when said compound bears a charge, the compound further comprises one or more counterions;

to a subject in need thereof more than 6 hours post ischemia onset.

In another aspect, the present invention may provide a method of treating ischemic injury comprising administering a therapeutically effective amount of a compound of formula (I):

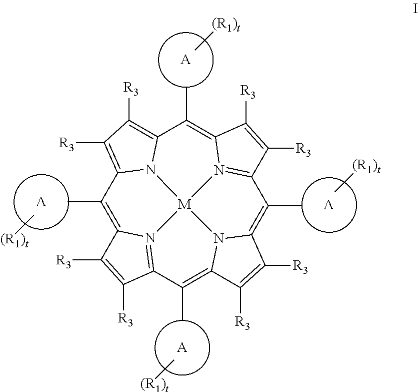

wherein:

each A is independently a heteroaryl group;

each R₁ is independently selected from H, C₆₋₁₂ alkyl, —(CH₂)ₙOR₂, —(CH₂)ₙSR₂, —(CH₂)ₙNR₂R₂, —(CH₂)ₙC(O)OR₄ and —(CH₂)ₘCHₚX_{q};

each R₂ is independently selected from hydrogen, alkyl, haloalkyl and —C(O)R₄;

each R₃ is independently selected from hydrogen, halogen, hydroxyl, alkoxy, alkyl, alkenyl, alkynyl, cycloalkyl, heterocycloalkyl, amino, amide, nitro, carboxylic acid, carboxyl, aryl, heteroaryl, thiol, thioalkyl, thioester, disulfide, phosphine, carbonyl, carbonylamino, formyl, sulfonyl, sulfonylamino, cyano, isocyano, C₁₋₄ alkyl aryl and C₁₋₄ alkyl heteroaryl;

each R₄ is independently selected from hydrogen, halogen, hydroxyl, alkoxy, alkyl, alkenyl, alkynyl, cycloalkyl, heterocycloalkyl, amino, amide, nitro, carboxylic acid, carboxyl, aryl, heteroaryl, thiol, thioalkyl, thioester, disulfide, phosphine, carbonyl, carbonylamino, formyl, sulfonyl, sulfonylamino, cyano, isocyano, C₁₋₄ alkyl aryl and C₁₋₄ alkyl heteroaryl;

each X is independently a halogen;

n is 1 to 12;

m is 1 to 11;

p is 0 to 3;

q is 0 to 3;

t is 0 to 2;

wherein p+q is 3; and

M is selected from Mn, Fe, Co, Ni, Cu, V or 2 hydrogens;

wherein when said compound bears a charge, the compound further comprises one or more counterions;

to a subject in need thereof at least once per day for at least 5 days post ischemia onset.

In another aspect, the present invention may provide a method of providing neuroprotection comprising administering a therapeutically effective amount of a compound of formula (I):

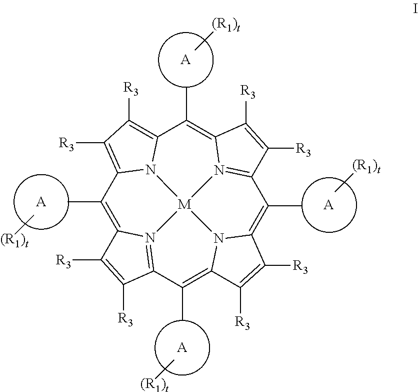

wherein:

each A is independently a heteroaryl group;

each R₁ is independently selected from H, C₆₋₁₂ alkyl, —(CH₂)ₙ0R₂, —(CH₂)ₙSR₂, —(CH₂)ₙNR₂R₂, —(CH₂)ₙC(O)OR₄ and —(CH₂)ₘCHₚX_{q};

each R₂ is independently selected from hydrogen, alkyl, haloalkyl and —C(O)R₄;

each R₃ is independently selected from hydrogen, halogen, hydroxyl, alkoxy, alkyl, alkenyl, alkynyl, cycloalkyl, heterocycloalkyl, amino, amide, nitro, carboxylic acid, carboxyl, aryl, heteroaryl, thiol, thioalkyl, thioester, disulfide, phosphine, carbonyl, carbonylamino, formyl, sulfonyl, sulfonylamino, cyano, isocyano, C₁₋₄ alkyl aryl and C₁₋₄ alkyl heteroaryl;

each R₄ is independently selected from hydrogen, halogen, hydroxyl, alkoxy, alkyl, alkenyl, alkynyl, cycloalkyl, heterocycloalkyl, amino, amide, nitro, carboxylic acid, carboxyl, aryl, heteroaryl, thiol, thioalkyl, thioester, disulfide, phosphine, carbonyl, carbonylamino, formyl, sulfonyl, sulfonylamino, cyano, isocyano, C₁₋₄ alkyl aryl and C₁₋₄ alkyl heteroaryl;

each X is independently a halogen;

n is 1 to 12;

m is 1 to 11;

p is 0 to 3;

q is 0 to 3;

t is 0 to 2;

wherein p+q is 3; and

M is selected from Mn, Fe, Co, Ni, Cu, V or 2 hydrogens;

wherein when said compound bears a charge, the compound further comprises one or more counterions;

to a subject in need thereof more than 4.5 hours post ischemia onset.

In another aspect, the present invention may provide a method of providing neuroprotection comprising administering a therapeutically effective amount of a compound of formula (I):

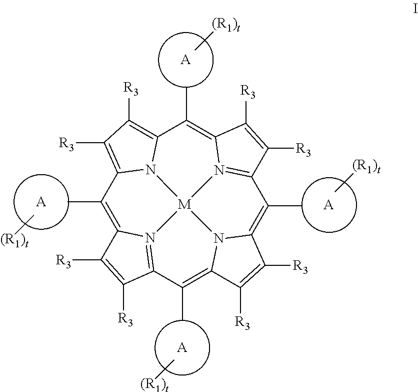

wherein:

each A is independently a heteroaryl group;

each R₁ is independently selected from H, C₆₋₁₂ alkyl, —(CH₂)ₙ0R₂, —(CH₂)ₙSR₂, —(CH₂)ₙNR₂R₂, —(CH₂)ₙC(O)OR₄ and —(CH₂)ₘCHₚX_{q};

each R₂ is independently selected from hydrogen, alkyl, haloalkyl and —C(O)R₄;

each R₃ is independently selected from hydrogen, halogen, hydroxyl, alkoxy, alkyl, alkenyl, alkynyl, cycloalkyl, heterocycloalkyl, amino, amide, nitro, carboxylic acid, carboxyl, aryl, heteroaryl, thiol, thioalkyl, thioester, disulfide, phosphine, carbonyl, carbonylamino, formyl, sulfonyl, sulfonylamino, cyano, isocyano, C₁₋₄ alkyl aryl and C₁₋₄ alkyl heteroaryl;

each R₄ is independently selected from hydrogen, halogen, hydroxyl, alkoxy, alkyl, alkenyl, alkynyl, cycloalkyl, heterocycloalkyl, amino, amide, nitro, carboxylic acid, carboxyl, aryl, heteroaryl, thiol, thioalkyl, thioester, disulfide, phosphine, carbonyl, carbonylamino, formyl, sulfonyl, sulfonylamino, cyano, isocyano, C₁₋₄ alkyl aryl and C₁₋₄ alkyl heteroaryl;

each X is independently a halogen;

n is 1 to 12;

m is 1 to 11;

p is 0 to 3;

q is 0 to 3;

t is 0 to 2;

wherein p+q is 3; and

M is selected from Mn, Fe, Co, Ni, Cu, V or 2 hydrogens;

wherein when said compound bears a charge, the compound further comprises one or more counterions;

to a subject in need thereof more than 6 hours post ischemia onset.

In another aspect, the present invention may provide a method of providing neuroprotection comprising administering a therapeutically effective amount of a compound of formula (I):

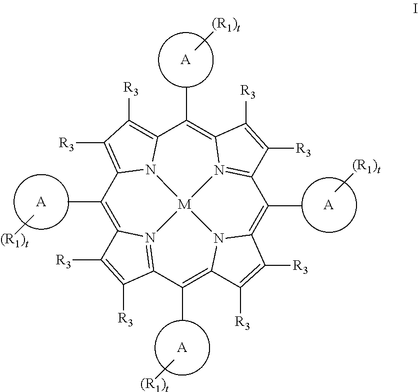

wherein:

each A is independently a heteroaryl group;

each R₁ is independently selected from H, C₆₋₁₂ alkyl, —(CH₂)ₙOR₂, —(CH₂)ₙSR₂, —(CH₂)ₙNR₂R₂, —(CH₂)ₙC(O)OR₄ and —(CH₂)ₘCHₚX_{q};

each R₂ is independently selected from hydrogen, alkyl, haloalkyl and —C(O)R₄;

each R₃ is independently selected from hydrogen, halogen, hydroxyl, alkoxy, alkyl, alkenyl, alkynyl, cycloalkyl, heterocycloalkyl, amino, amide, nitro, carboxylic acid, carboxyl, aryl, heteroaryl, thiol, thioalkyl, thioester, disulfide, phosphine, carbonyl, carbonylamino, formyl, sulfonyl, sulfonylamino, cyano, isocyano, C₁₋₄ alkyl aryl and C₁₋₄ alkyl heteroaryl;

each R₄ is independently selected from hydrogen, halogen, hydroxyl, alkoxy, alkyl, alkenyl, alkynyl, cycloalkyl, heterocycloalkyl, amino, amide, nitro, carboxylic acid, carboxyl, aryl, heteroaryl, thiol, thioalkyl, thioester, disulfide, phosphine, carbonyl, carbonylamino, formyl, sulfonyl, sulfonylamino, cyano, isocyano, C₁₋₄ alkyl aryl and C₁₋₄ alkyl heteroaryl;

each X is independently a halogen;

n is 1 to 12;

m is 1 to 11;

p is 0 to 3;

q is 0 to 3;

t is 0 to 2;

wherein p+q is 3; and

M is selected from Mn, Fe, Co, Ni, Cu, V or 2 hydrogens;

wherein when said compound bears a charge, the compound further comprises one or more counterions;

to a subject in need thereof at least once per day for at least 5 days post ischemia onset.

In another aspect, the present invention may provide a method of treating subarachnoid hemorrhage comprising administering a therapeutically effective amount of a compound of formula (I):

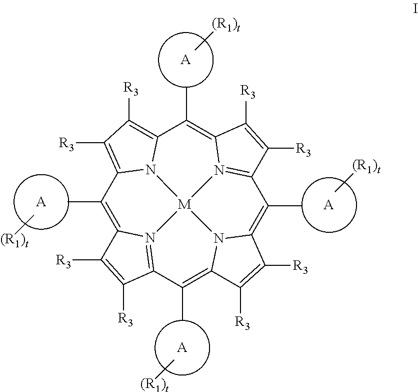

wherein:

each A is independently a heteroaryl group;

each R₁ is independently selected from H, C₆₋₁₂ alkyl, —(CH₂)ₙ0R₂, —(CH₂)ₙSR₂, —(CH₂)ₙNR₂R₂, —(CH₂)ₙC(O)OR₄ and —(CH₂)ₘCHₚX_{q};

each R₂ is independently selected from hydrogen, alkyl, haloalkyl and —C(O)R₄;

each R₃ is independently selected from hydrogen, halogen, hydroxyl, alkoxy, alkyl, alkenyl, alkynyl, cycloalkyl, heterocycloalkyl, amino, amide, nitro, carboxylic acid, carboxyl, aryl, heteroaryl, thiol, thioalkyl, thioester, disulfide, phosphine, carbonyl, carbonylamino, formyl, sulfonyl, sulfonylamino, cyano, isocyano, C₁₋₄ alkyl aryl and C₁₋₄ alkyl heteroaryl;

each R₄ is independently selected from hydrogen, halogen, hydroxyl, alkoxy, alkyl, alkenyl, alkynyl, cycloalkyl, heterocycloalkyl, amino, amide, nitro, carboxylic acid, carboxyl, aryl, heteroaryl, thiol, thioalkyl, thioester, disulfide, phosphine, carbonyl, carbonylamino, formyl, sulfonyl, sulfonylamino, cyano, isocyano, C₁₋₄ alkyl aryl and C₁₋₄ alkyl heteroaryl;

each X is independently a halogen;

n is 1 to 12;

m is 1 to 11;

p is 0 to 3;

q is 0 to 3;

t is 0 to 2;

wherein p+q is 3; and

M is selected from Mn, Fe, Co, Ni, Cu, V or 2 hydrogens;

wherein when said compound bears a charge, the compound further comprises one or more counterions;

to a subject in need thereof.

In another aspect, the present invention may provide a method of treating traumatic brain injury (TBI) comprising administering a therapeutically effective amount of a compound of formula (I):

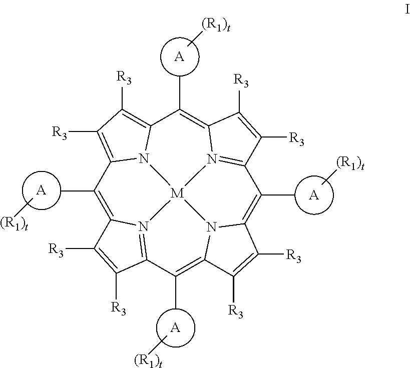

wherein:

each A is independently a heteroaryl group;

each R₁ is independently selected from H, C₆₋₁₂ alkyl, —(CH₂)ₙ0R₂, —(CH₂)ₙSR₂, —(CH₂)ₙNR₂R₂, —(CH₂)ₙC(O)OR₄ and —(CH₂)ₘCHₚX_{q};

each R₂ is independently selected from hydrogen, alkyl, haloalkyl and —C(O)R₄;

each R₃ is independently selected from hydrogen, halogen, hydroxyl, alkoxy, alkyl, alkenyl, alkynyl, cycloalkyl, heterocycloalkyl, amino, amide, nitro, carboxylic acid, carboxyl, aryl, heteroaryl, thiol, thioalkyl, thioester, disulfide, phosphine, carbonyl, carbonylamino, formyl, sulfonyl, sulfonylamino, cyano, isocyano, C₁₋₄ alkyl aryl and C₁₋₄ alkyl heteroaryl;

each R₄ is independently selected from hydrogen, halogen, hydroxyl, alkoxy, alkyl, alkenyl, alkynyl, cycloalkyl, heterocycloalkyl, amino, amide, nitro, carboxylic acid, carboxyl, aryl, heteroaryl, thiol, thioalkyl, thioester, disulfide, phosphine, carbonyl, carbonylamino, formyl, sulfonyl, sulfonylamino, cyano, isocyano, C₁₋₄ alkyl aryl and C₁₋₄ alkyl heteroaryl;

each X is independently a halogen;

n is 1 to 12;

m is 1 to 11;

p is 0 to 3;

q is 0 to 3;

t is 0 to 2;

wherein p+q is 3; and

M is selected from Mn, Fe, Co, Ni, Cu, V or 2 hydrogens;

wherein when said compound bears a charge, the compound further comprises one or more counterions;

to a subject in need thereof.

In another aspect, the present invention may provide a method of treating spinal cord injury (SCI) comprising administering a therapeutically effective amount of a compound of formula (I):

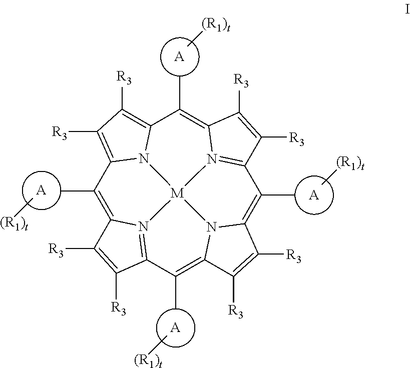

wherein:

each A is independently a heteroaryl group;

each R₁ is independently selected from H, C₆₋₁₂ alkyl, —(CH₂)ₙOR₂, —(CH₂)ₙSR₂, —(CH₂)ₙNR₂R₂, —(CH₂)ₙC(O)OR₄ and —(CH₂)ₘCHₚX_{q};

each R₂ is independently selected from hydrogen, alkyl, haloalkyl and —C(O)R₄;

each R₃ is independently selected from hydrogen, halogen, hydroxyl, alkoxy, alkyl, alkenyl, alkynyl, cycloalkyl, heterocycloalkyl, amino, amide, nitro, carboxylic acid, carboxyl, aryl, heteroaryl, thiol, thioalkyl, thioester, disulfide, phosphine, carbonyl, carbonylamino, formyl, sulfonyl, sulfonylamino, cyano, isocyano, C₁₋₄ alkyl aryl and C₁₋₄ alkyl heteroaryl;

each R₄ is independently selected from hydrogen, halogen, hydroxyl, alkoxy, alkyl, alkenyl, alkynyl, cycloalkyl, heterocycloalkyl, amino, amide, nitro, carboxylic acid, carboxyl, aryl, heteroaryl, thiol, thioalkyl, thioester, disulfide, phosphine, carbonyl, carbonylamino, formyl, sulfonyl, sulfonylamino, cyano, isocyano, C₁₋₄ alkyl aryl and C₁₋₄ alkyl heteroaryl;

each X is independently a halogen;

n is 1 to 12;

m is 1 to 11;

p is 0 to 3;

q is 0 to 3;

t is 0 to 2;

wherein p+q is 3; and

M is selected from Mn, Fe, Co, Ni, Cu, V or 2 hydrogens;

wherein when said compound bears a charge, the compound further comprises one or more counterions;

to a subject in need thereof.

### BRIEF DESCRIPTION OF THE DRAWINGS

FIG. 1 shows neurologic scores at 72 hrs post-SAH and treatment with saline or MnTnHex-2-PyP⁵⁺.

FIG. 2 shows right anterior cerebral artery diameters 72 hrs post-SAH and treatment with saline or MnTnHex-2-PyP⁵⁺.

FIG. 3 shows neurologic scores 7 days after 90 min MCAO and treatment with twice daily injections of MnTnHex-2-PyP⁵⁺ for 7 days, beginning 5 min after reperfusion onset.

FIG. 4 shows measurements of infarct volumes measured 7 days after 90 min MCAO and treatment with MnTnHex-2-PyP⁵⁺ twice a day for 7 days, beginning 5 min after reperfusion onset.

FIG. 5 shows neurologic scores 7 days after 90 min MCAO and treatment with MnTnHex-2-PyP⁵⁺ twice a day for 7 days, beginning 6 h after reperfusion onset.

FIG. 6 shows measurements of infarct volumes measured 7 days after 90 min MCAO and treatment with MnTnHex-2-PyP⁵⁺ twice a day for 7 days, beginning 6 h after reperfusion onset.

FIG. 7 shows an electrophoretic mobility shift assay (EMSA) on nuclear extracts isolated from ischemic brains of rats subjected to 90 min MCAO and then treated with vehicle or MnTnHex-2-PyP⁵⁺, and an immunoblot of the same samples.

FIG. 8 shows measurements of TNF-α and IL-6 from rat brains following treatment with vehicle or MnTnHex-2-PyP⁵⁺ at 12 and 18 hours post-MCAO.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

The present invention generally provides methods of treating ischemic injury or subarachnoid hemorrhage comprising administering a therapeutically effective amount of a substituted porphyrin to a subject in need thereof.

### DEFINITIONS

“Acyl” or “carbonyl” refers to the group —C(O)R wherein R is alkyl, alkenyl, alkynyl, aryl, heteroaryl, carbocyclic, heterocarbocyclic, C₁₋₄ alkyl aryl or C₁₋₄ alkyl heteroaryl. C₁₋₄ alkylcarbonyl refers to a group wherein the carbonyl moiety is preceded by an alkyl chain of 1-4 carbon atoms.

“Alkenyl” refers to an unsaturated aliphatic hydrocarbon moiety including straight chain and branched chain groups. Alkenyl moieties must contain at least one double bond. Suitably, an alkenyl moiety has from 2 to 10 carbon atoms. In some embodiments, the alkenyl has no more than 8 carbons or no more than 5 carbons or at least 3 carbons. “Alkenyl” may be exemplified by groups such as ethenyl, n-propenyl, isopropenyl, n-butenyl and the like. Alkenyl groups may be substituted or unsubstituted or branched or unbranched. More than one substituent may be present. Substituents may also be themselves substituted. Substituents can be placed on the alkene itself and also on the adjacent member atoms or the alkenyl moiety. “C₂₋₄ alkenyl” refers to alkenyl groups containing two to four carbon atoms.

“Alkoxy” refers to the group —O—R wherein R is acyl, alkyl alkenyl, alkyl alkynyl, aryl, carbocyclic, heterocarbocyclic, heteroaryl, C₁₋₄ alkyl aryl or C₁₋₄ alkyl heteroaryl. The R group itself may be further substituted.

“Alkyl” refers to a monovalent alkyl group, such as methyl, ethyl, propyl, etc. In some embodiments, the alkyl has from 1 to 10 carbon atoms. In other embodiments, the alkyl has no more than 8 carbon atoms or no more than 6 carbon atoms. In other embodiments, the alkyl group has at least 3 carbon atoms. The alkyl group can be saturated or unsaturated, branched or unbranched, and substituted or unsubstituted. Substituents may also be substituted. “Lower alkyl” refers to an alkyl group with from 1 to 4 carbon atoms.

“Alkylene” refers to a divalent alkyl group, such as methylene (—CH₂—), ethylene (—CH₂—CH₂—), propylene (—CH₂—CH₂—CH₂—), etc. In some embodiments, the alkylene has from 1 to 10 carbon atoms. In other embodiments, the alkylene has no more than 8 carbon atoms or no more than 6 carbon atoms. In further embodiments, the alkylene group has at least 3 carbon atoms. In some embodiments, the alkylene group has from 3 to 6 carbon atoms. In some embodiments, one or more of the carbon atoms is replaced by a heteroatom. The alkylene group may be saturated or unsaturated. The alkylene group may suitably be branched and in some embodiments, the branched alkylene group forms a carbocycle or aryl group. In addition, the alkylene group may be substituted.

“Alkynyl” refers to an unsaturated aliphatic hydrocarbon moiety including straight chain and branched chain groups. Alkynyl moieties must contain at least one triple bond. Alkynyl moieties suitably have from 2 to 10 carbons. In some embodiments, the alkynyl has no more than 8 carbons or no more than 5 carbons or at least 3 carbons. “Alkynyl” may be exemplified by groups such as ethynyl, propynyl, n-butynyl and the like. Alkynyl groups may be substituted or unsubstituted or branched or unbranched. More than one substituent may be present. Substituents may also be themselves substituted. Substituents are not on the alkyne itself but on the adjacent member atoms of the alkynyl moiety. “C₂₋₄ alkynyl” refers to alkynyl groups containing two to four carbon atoms.

“Amino” refers to the group —NR′R′ wherein each R′ is, independently, hydrogen, amino, hydroxyl, alkoxyl, alkyl, aryl, cycloalkyl, heterocycloalkyl, heteroaryl, C₁₋₄ alkyl aryl or C₁₋₄ alkyl heteroaryl. The two R′ groups may themselves be linked to form a ring. The R′ groups may themselves be further substituted.

“Aryl” refers to an aromatic carbocyclic group. Suitably, aryl has 5 to 10 carbons and may be monocyclic or bicyclic. In some embodiments, the aryl group has 5 to 6 carbons and in other embodiments, the aryl group may have 9 to 10 carbons. “Aryl” may be exemplified by phenyl or naphthalene or cyclopentadienyl. The aryl group may be substituted or unsubstituted. More than one substituent may be present. Substituents may also be themselves substituted. When substituted, the substituent group is preferably but not limited to heteroaryl, acyl, carboxyl, carbonylamino, nitro, amino, cyano, halogen, or hydroxyl.

“Carboxyl” refers to the group —C(═O)O—R, wherein each R is, independently, hydrogen, alkyl, aryl, cycloalkyl, heterocycloalkyl, heteroaryl, C₁₋₄ alkyl aryl or C₁₋₄ alkyl heteroaryl.

“Carbonyl” refers to the group —C(O)R wherein each R is, independently, hydrogen, alkyl, aryl, cycloalkyl, heterocycloalkyl, heteroaryl, C₁₋₄ alkyl aryl or C₁₋₄ alkyl heteroaryl.

“Carbonylamino” refers to the group —C(O)NR′R′ wherein each R′ is, independently, hydrogen, alkyl, aryl, cycloalkyl, heterocycloalkyl, heteroaryl, C₁₋₄ alkyl aryl or C₁₋₄ alkyl heteroaryl. The two R′ groups may themselves be linked to form a ring.

“C₁₋₄ alkyl aryl” refers to C₁₋₄ alkyl groups having an aryl substituent such that the aryl substituent is bonded through an alkyl group. “C₁₋₄ alkyl aryl” may be exemplified by benzyl.

“C₁₋₄ alkyl heteroaryl” refers to C₁₋₄ alkyl groups having a heteroaryl substituent such that the heteroaryl substituent is bonded through an alkyl group.

“C₆₋₁₂ alkyl” refers to alkyl groups having from 6 to 12 carbon atoms. Suitable groups include hexyl, heptyl, octyl, etc.

“Carbocyclic group” or “cycloalkyl” means a monovalent saturated or unsaturated hydrocarbon ring. Carbocyclic groups are monocyclic, or are fused, spiro, or bridged bicyclic ring systems. Monocyclic carbocyclic groups contain 3 to 10 carbon atoms, suitably 4 to 7 carbon atoms, or 5 to 6 carbon atoms in the ring. Bicyclic carbocyclic groups contain 8 to 12 carbon atoms, suitably 9 to 10 carbon atoms in the ring. Carbocyclic groups may be substituted or unsubstituted. More than one substituent may be present. Substituents may also be themselves substituted. Suitable carbocyclic groups include cyclopropyl, cyclobutyl, cyclopentyl, cyclohexyl, cyclohexenyl, and cycloheptyl. Carbocyclic groups are not aromatic.

“Counterion” refers to any chemically compatible species used for charge balance. A counterion may be a positively charged cation or negatively charged anion. Exemplary counteranions include, but are not limited to, chloride, bromide, iodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate, pamoate, hexafluorophosphate, tetrafluoroborate, tetraphenyl borate, perchlorate, trifluoromethanesulfonate or hexafluoroantimonate.

“Disulfide” refers to the group —S—S—R, wherein R is alkyl, aryl, heteroaryl, C₁₋₄ alkyl aryl or C₁₋₄ alkyl heteroaryl.

“Halogen” refers to a fluoro, chloro, iodo or bromo.

“Heteroalkyl” refers to an alkyl group containing one or more heteroatoms.

“Heteroaryl” refers to a 5 or 10 membered aromatic ring which contains 1 or more heteroatoms. Suitably, the heteroaryl group has 5 to 6 members or 9 to 10 members. If more than one heteroatom is present, the heteroatoms may be the same or different. The heteroaryl groups are optionally substituted. In some embodiments, the heteroaryl group has a nitrogen at the ortho position. In some embodiments, the heteroaryl group has anitrogen at the meta position. Suitably, the heteroaryl group has 1 nitrogen, 2 nitrogens or 3 nitrogens, such as pyridyl, imidazolyl, pyrazolyl, pyrimidyl and thiazolyl.

“Heteroatom” refers to a nitrogen, sulfur or oxygen. The heteroatom may be substituted in some embodiments. Groups containing more than one heteroatom may contain different heteroatoms.

“Heterocarbocyclic group” or “heterocycloalkyl” or “heterocyclic” means a monovalent saturated or unsaturated hydrocarbon ring containing at least one heteroatom. Heterocarbocyclic groups are monocyclic, or are fused, spiro, or bridged bicyclic ring systems. Monocyclic heterocarbocyclic groups contain 3 to 10 carbon atoms, suitably 4 to 7 carbon atoms, or 5 to 6 carbon atoms in the ring. Bicyclic heterocarbocyclic groups contain 8 to 12 carbon atoms, suitably 9 to 10 carbon atoms in the ring. Heterocarbocyclic groups may be substituted or unsubstituted. More than one substituent may be present. Substituents may also be themselves substituted. Suitable heterocarbocyclic groups include epoxy, tetrahydrofuranyl, azacyclopentyl, azacyclohexyl, piperidyl, and homopiperidyl. Heterocarbocyclic groups are not aromatic.

“Hydroxy” or “hydroxyl” means a chemical entity that consists of —OH. Alcohols contain hydroxy groups. Hydroxy groups may be free or protected. An alternative name for hydroxy is hydroxyl.

“Member atom” means a carbon, nitrogen, oxygen or sulfur atom. Member atoms may be substituted up to their normal valence. If substitution is not specified the substituents required for valency are hydrogen.

“Ring” means a collection of member atoms that are cyclic. Rings may be carbocyclic, aromatic, or heterocyclic or heteroaromatic, and may be substituted or unsubstituted, and may be saturated or unsaturated. More than one substituent may be present. Ring junctions with the main chain may be fused or spirocyclic. Rings may be monocyclic or bicyclic. Rings contain at least 3 member atoms and at most 10 member atoms. Monocyclic rings may contain 3 to 7 member atoms and bicyclic rings may contain from 8 to 12 member atoms. Bicyclic rings themselves may be fused or spirocyclic.

“Sulfonyl” refers to the —S(O)₂R′ group wherein R′ is alkoxy, alkyl, aryl, carbocyclic, heterocarbocyclic, heteroaryl, C₁₋₄ alkyl aryl or C₁₋₄ alkyl heteroaryl.

“Sulfonylamino” refers to the —S(O)₂NR′R′ group wherein each R′ is independently alkyl, aryl, heteroaryl, C₁₋₄ alkyl aryl or C₁₋₄ alkyl heteroaryl.

“Thioalkyl” refers to the group —S-alkyl.

“Thiol” refers to the group —SH.

Suitable substituents include, but are not limited to halogen, hydroxyl, alkoxy, haloalkoxy, thioalkyl, alkyl, alkenyl, alkynyl, cycloalkyl, heterocycloalkyl, amino, amide, nitro, keto, oxo, carboxylic acid, carboxyl, aryl, heteroaryl, thiol, thioalkyl, thioester, disulfide, phosphine, carbonyl, carbonylamino, formyl, sulfonyl, sulfonylamino, cyano, isocyano, C₁₋₄ alkyl aryl and C₁₋₄ alkyl heteroaryl.

### Substituted Porphyrins

The invention features methods of treating ischemic injury comprising administering, to a subject in need thereof, a therapeutically effective amount of a substituted porphyrin compound. The invention also features methods of providing neuroprotection, methods of treating subarachnoid hemorrhage, methods of treating traumatic brain injury and methods of treating spinal cord injury using substituted porphyrins.

The substituted porphyrins include compounds of formula (I):

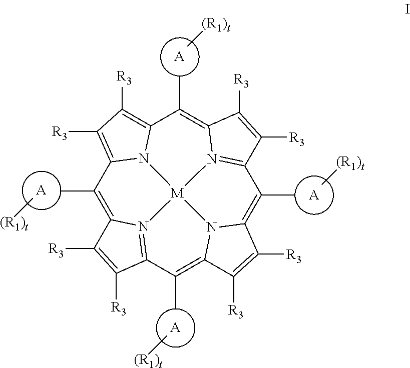

wherein:

each A is independently a heteroaryl group;

each R₁ is independently selected from H, C₆₋₁₂ alkyl, —(CH₂)ₙOR₂, —(CH₂)ₙSR₂, —(CH₂)ₙNR₂R₂, —(CH₂)ₙC(O)OR₄ and —(CH₂)ₘCHₚX_{q};

each R₂ is independently selected from hydrogen, alkyl, haloalkyl and —C(O)R₄;

each R₃ is independently selected from hydrogen, halogen, hydroxyl, alkoxy, alkyl, alkenyl, alkynyl, cycloalkyl, heterocycloalkyl, amino, amide, nitro, carboxylic acid, carboxyl, aryl, heteroaryl, thiol, thioalkyl, thioester, disulfide, phosphine, carbonyl, carbonylamino, formyl, sulfonyl, sulfonylamino, cyano, isocyano, C₁₋₄ alkyl aryl and C₁₋₄ alkyl heteroaryl;

each R₄ is independently selected from hydrogen, halogen, hydroxyl, alkoxy, alkyl, alkenyl, alkynyl, cycloalkyl, heterocycloalkyl, amino, amide, nitro, carboxylic acid, carboxyl, aryl, heteroaryl, thiol, thioalkyl, thioester, disulfide, phosphine, carbonyl, carbonylamino, formyl, sulfonyl, sulfonylamino, cyano, isocyano, C₁₋₄ alkyl aryl and C₁₋₄ alkyl heteroaryl;

each X is independently a halogen;

n is 1 to 12;

m is 1 to 11;

p is 0 to 3;

q is 0 to 3;

t is 0 to 2;

wherein p+q is 3; and

M is selected from Mn, Fe, Co, Ni, Cu, V or 2 hydrogens;

wherein when said compound bears a charge, the compound further comprises one or more counterions.

In some embodiments, R₁ is a substituted C₆₋₁₂ alkyl group. In some embodiments, R₁ is a C₆₋₁₂ alkyl group substituted with a hydroxy, alkoxy, thioalkoxy or haloalkoxy substituent. In some embodiments, R₁ is a C₆ alkyl group (e.g., n-hexyl). In some embodiments, R₁ is a C₈ alkyl group (e.g., n-octyl). In some embodiments, R₁ is a C₉ alkyl group (e.g., n-nonyl). In some embodiments, R₁ is a C₁₋₂ alkyl group (e.g., n-dodecyl).

In some embodiments, A is pyridyl. Suitable compounds according to formula (I) include 2-pyridyl (ortho), 3-pyridyl (meta) and 4-pyridyl (para) substituted porphyrins, such as those illustrated below:

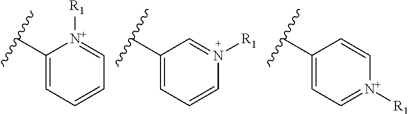

In some embodiments, A is imidazolyl. In some embodiments, A is thiazolyl. In some embodiments, A is pyrazolyl. In some embodiments, A is pyrimidyl.

Suitable Ris include —(CH₂)ₙOR₂, —(CH₂)ₙSR₂, —(CH₂)ₙNR₂R₂, —(CH₂)ₙC(O)OR₄, —(CH₂)ₘCHₚX_{q}, wherein each R₂ is independently selected from hydrogen, alkyl (e.g., methyl, ethyl, t-butyl or isopropyl), haloalkyl (e.g., trifluoromethyl or trifluoroethyl), or —C(O)R₄; X is a halogen, such as F, Cl or Br; R₄ is hydrogen, halogen, hydroxyl, alkoxy, alkyl, alkenyl, alkynyl, cycloalkyl, heterocycloalkyl, amino, amide, nitro, carboxylic acid, carboxyl, aryl, heteroaryl, thiol, thioalkyl, thioester, disulfide, phosphine, carbonyl, carbonylamino, formyl, sulfonyl, sulfonylamino, cyano, isocyano, C₁ alkyl aryl or C₁ alkyl heteroaryl; n is 1 to 12; m is 1 to 11; p is 0 to 3 and q is 0 to 3, wherein p+q is 3. In some embodiments, R₂ is an oxygen protecting group or a nitrogen protecting group, such as those typically used in the art. In some embodiments, n is 1, 2, 3, 4, 5, 6, 7 or 8 or 9. In other embodiments, m is 1, 2, 3, 4, 5, 6, 7 or 8.

For example, R₁ may be —(CH₂)₅CH₃, —(CH₂)₈CH₃, —(CH₂)₂OCH₃, —(CH₂)₆OCH₃, —(CH₂)₆OCH₂CH₃, —(CH₂)₆OCH(CH₃)₂, —(CH₂)₆OC(CH₃)₃, —(CH₂)₆OCF₃, —(CH₂)₆OCH₂CF₃, —(CH₂)₆OH, —(CH₂)₂SCH₃, —(CH₂)₆SCH₃, —(CH₂)₆NH₂, —(CH₂)₅CH₂F, —(CH₂)₅CHF₂, or —(CH₂)₅CF₃.

The substituted porphyrins also include compounds of formula (II):

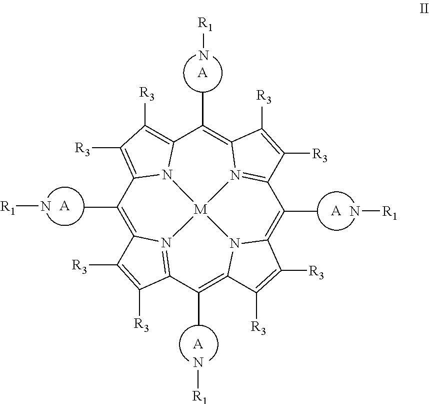

wherein:

each A is independently a heteroaryl group;

each R₁ is independently selected from H, C₆₋₁₂ alkyl, —(CH₂)ₙOR₂, —(CH₂)ₙSR₂, —(CH₂)ₙNR₂R₂, —(CH₂)ₙC(O)OR₄ and —(CH₂)ₘCHₚX_{q};

each R₂ is independently selected from hydrogen, alkyl, haloalkyl and —C(O)R₄;

each R₃ is independently selected from hydrogen, halogen, hydroxyl, alkoxy, alkyl, alkenyl, alkynyl, cycloalkyl, heterocycloalkyl, amino, amide, nitro, carboxylic acid, carboxyl, aryl, heteroaryl, thiol, thioalkyl, thioester, disulfide, phosphine, carbonyl, carbonylamino, formyl, sulfonyl, sulfonylamino, cyano, isocyano, C₁₋₄ alkyl aryl and C₁₋₄ alkyl heteroaryl;

each R₄ is independently selected from hydrogen, halogen, hydroxyl, alkoxy, alkyl, alkenyl, alkynyl, cycloalkyl, heterocycloalkyl, amino, amide, nitro, carboxylic acid, carboxyl, aryl, heteroaryl, thiol, thioalkyl, thioester, disulfide, phosphine, carbonyl, carbonylamino, formyl, sulfonyl, sulfonylamino, cyano, isocyano, C₁₋₄ alkyl aryl and C₁₋₄ alkyl heteroaryl;

each X is independently a halogen;

n is 1 to 12;

m is 1 to 11;

p is 0 to 3;

q is 0 to 3;

t is 0 to 2;

wherein p+q is 3; and

M is selected from Mn, Fe, Co, Ni, Cu, V or 2 hydrogens;

wherein when said compound bears a charge, the compound further comprises one or more counterions.

In some embodiments, in the compound of formula (II):

each A is independently a pyridyl group;

each R₁ is independently H or C₆₋₁₂ alkyl;

each R₃ is independently selected from hydrogen, halogen, hydroxyl, alkoxy, alkyl, alkenyl, alkynyl, cycloalkyl, heterocycloalkyl, amino, amide, nitro, carboxylic acid, carboxyl, aryl, heteroaryl, thiol, thioalkyl, thioester, disulfide, phosphine, carbonyl, carbonylamino, formyl, sulfonyl, sulfonylamino, cyano, isocyano, C₁₋₄ alkyl aryl and C₁₋₄ alkyl heteroaryl; and

M is selected from Mn, Fe, Co, Ni, Cu, V or 2 hydrogens.

In some embodiments, the substituted porphyrin is of one of the following formulae:

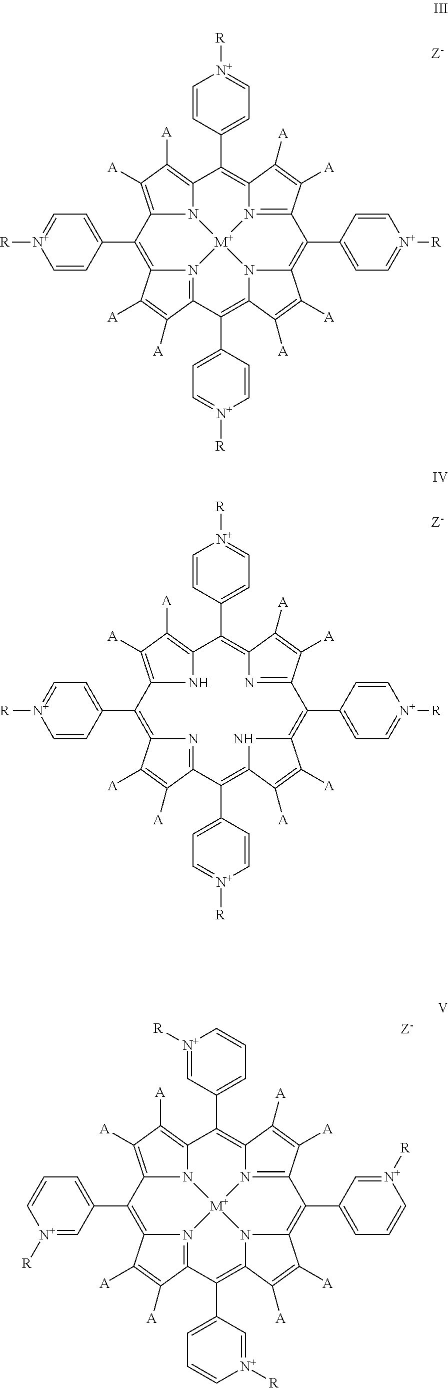

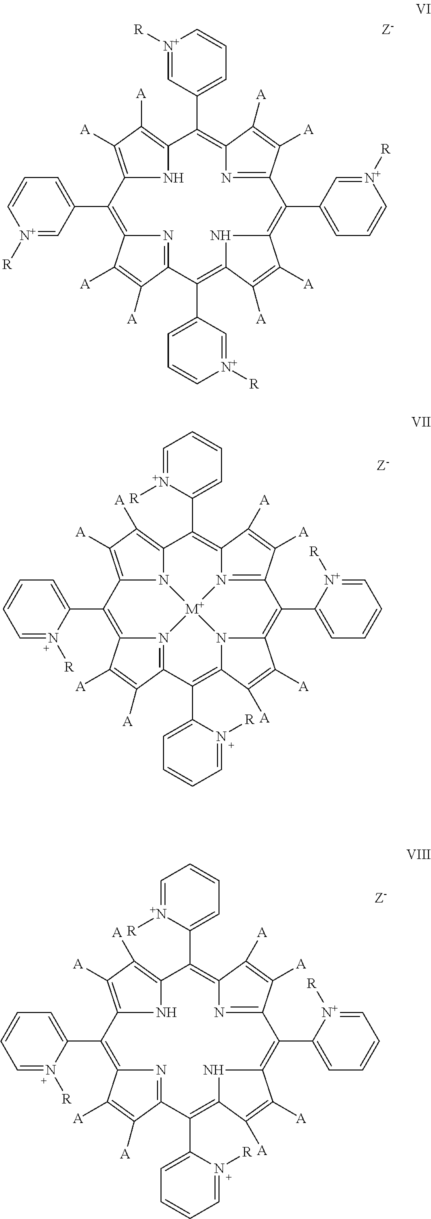

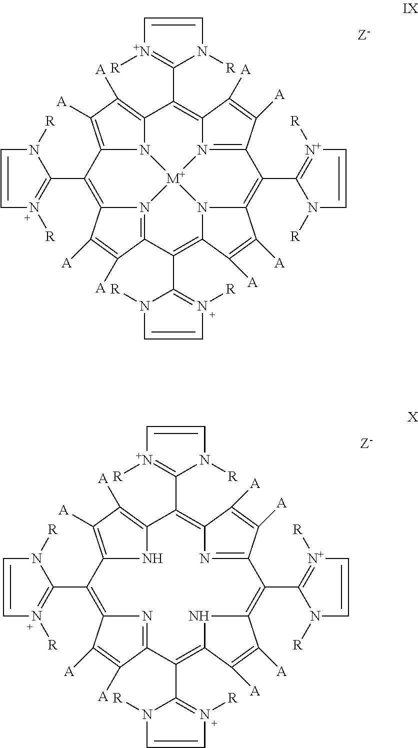

wherein:

when the compound is of Formula III-VIII, each R is, independently, —(CH₂)ₘCH₂OX or —(CH₂CH₂O)ₙX,

wherein:

m is 1-6,

n is 3-50, and

X is C₁₋₁₂ alkyl (straight chain or branched);

when the compound is of Formula IX or X, at least one R on each imidazole ring is, independently, —(CH₂)ₘCH₂OX or —(CH₂CH₂O)ₙX, the other R being, independently, a C₁₋₁₂ alkyl (straight chain or branched),

wherein

m is 1-6,

n is 3-50,

X is C₁₋₁₂ alkyl (straight chain or branched),

when the compound is any of Formulas III-X, each A is, independently, hydrogen or an electron withdrawing group,

M is metal selected from the group consisting of manganese, iron, copper, cobalt, nickel and zinc, and

Z⁻ is a counterion.

In some embodiments, the substituted porphyrin is of the following formula (XI):

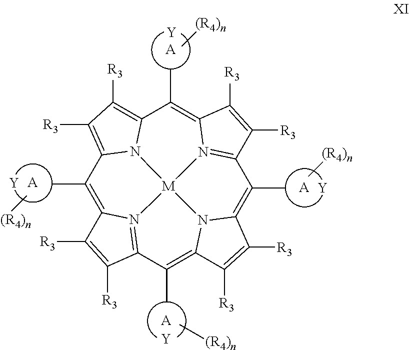

wherein each A is independently selected from the group consisting of an unsubstituted or substituted heteroaryl group and aryl group;

wherein each Y is independently selected from the group consisting of a CH and a heteroatom;

wherein each R₄ is independently —R₁—X—R₂;

wherein each R₁ is independently an unsubstituted or substituted alkylene;

wherein each X is independently selected from the group consisting of a direct bond and a heteroatom;

wherein each R₂ and R₃ are independently selected from the group consisting of hydrogen, halogen, hydroxyl, alkoxy, alkyl, alkenyl, alkynyl, cycloalkyl, heterocycloalkyl, amino, amide, nitro, keto, oxo, carboxylic acid, carboxyl, aryl, heteroaryl, thiol, thioalkyl, thioester, disulfide, phosphine, carbonyl, carbonylamino, formyl, sulfonyl, sulfonylamino, cyano, isocyano, C₁₋₄ alkyl aryl, and C₁₋₄ alkyl heteroaryl;

wherein each n is independently 0 to 2;

wherein M is selected from the group consisting of Mn, Fe, Co, Ni, Cu, V, and 2 hydrogens;

wherein at least one —R₁—X—R₂ contains at least one heteroatom; and

wherein at least one Y is N—R₄.

Exemplary porphyrins include:
Mn(III) 5,10,15,20-tetrakis(N-methylpyridinium-2-yl)porphyrin
Mn(III) 5,10,15,20-tetrakis(N-ethylpyridinium-2-yl)porphyrin
Mn(III) 5,10,15,20-tetrakis(N-n-propylpyridinium-2-yl)porphyrin
Mn(III) 5,10,15,20-tetrakis(N-n-butylpyridinium-2-yl)porphyrin
Mn(III) 5,10,15,20-tetrakis(N-n-hexylpyridinium-2-yl)porphyrin
Mn(III) 5,10,15,20-tetrakis(N-n-octylpyridinium-2-yl)porphyrin
Mn(III) 5,10,15,20-tetrakis[N,N′-diethylimidazolium-2-yl]porphyrin
Mn(III) tetrakis 5,10,15,20-tetrakis[N-(2-methoxyethyl)pyridinium-2-yl]porphyrin
Mn(III) tetrakis 5,10,15,20-tetrakis[N-methyl-N′-(2-methoxyethyl)imidazolium-2-yl]porphyrin
Mn(III) tetrakis 5,10,15,20-tetrakis[N,N′-di(2-methoxyethyl)imidazolium-2-yl]porphyrin

It may be convenient or desirable to prepare, purify, and/or handle the active compound in a chemically protected form. The term “chemically protected form”, as used herein, pertains to a compound in which one or more reactive functional groups are protected from undesirable chemical reactions, that is, are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, Protective Groups in Organic Synthesis (T. Green and P. Wuts, Wiley, 1999).

For example, an oxygen protecting group may be a hydroxy protecting group. A hydroxy group may be protected as an ether (—OR) or an ester (—OC(═O)R), for example, as: a t-butyl ether; a benzyl, benzhydryl(diphenylmethyl), or trityl(triphenylmethyl)ether; a trimethylsilyl or t-butyldimethylsilyl ether; or an acetyl ester (—OC(═O)CH₃, —OAc). For example, a nitrogen protecting group may be an amino protecting group. An amine group may be protected, for example, as an amide or a urethane, for example, as: a methyl amide (—NHCO—CH₃); a benzyloxy amide (—NHCO—OCH₂C₆H₅, —NHCbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH-Fmoc), as a 6-nitroveratryloxy amide (—NH—Nvoc), as a 2-trimethylsilylethyloxy amide (—NH-Teoc), as a 2,2,2-trichloroethyloxy amide (—NH-Troc), as an allyloxy amide (—NH-Alloc), as a 2(-phenylsulphonyl)ethyloxy amide (—NH—Psec); or, in suitable cases, as an N-oxide.

For example, an aldehyde or ketone group may be protected as an acetal or ketal, respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid. For example, a carboxylic acid group may be protected as an ester for example, as: an C₁₋₇ alkyl ester (e.g. a methyl ester; a t-butyl ester); a C₁₋₇ haloalkyl ester (e.g., a C₁₋₇ trihaloalkylester); a triC₁₋₇ alkylsilyl-C₁₋₇ alkyl ester; or a C₅₋₂₀ aryl-C₁₋₇ alkyl ester (e.g. a benzyl ester; a nitrobenzyl ester); or as an amide, for example, as a methyl amide. For example, a thiol group may be protected as a thioether (—SR), for example, as: a benzyl thioether; an acetamidomethyl ether (—S—CH₂NHC(═O)CH₃).

Other exemplary substituted porphyrins are described in WO 2005/077269, US 2008/0021007 and PCT/US2010/020328, the entire contents of each of which are hereby incorporated by reference.

### Synthesis of Substituted Porphyrins

The substituted porphyrins of the present invention may be synthesized in several steps. For example, for an ortho isomeric substituted Mn pyridylporphyrins, in a first step an aldehyde and a pyrrole may be condensed in a heated carboxylic acid, such as propionic acid at 130° C., to give a metal-free non-substituted porphyrinogen which in the presence of oxidant (H₂O₂ or O₂) is oxidized to porphyrin.

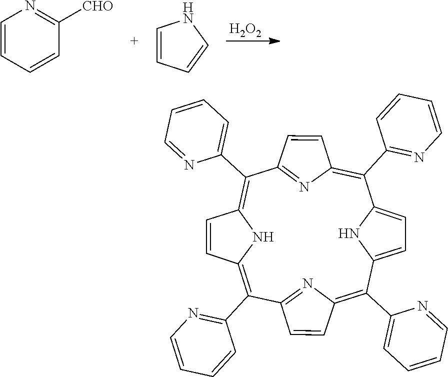

The product, H₂T-2-PyP may be purified by chromatography using a dichloromethane/methanol solvent system and is then forwarded to a second step where the pyridyl nitrogens are derivatized with appropriate side chains. For example, the pyridyl nitrogen may be derivatized with an alkyl group such as hexyl. In one such method, the derivatization/quaternization may occurs at ˜100° C. for a certain time period with p-alkyl- (or derivatized alkyl) toluenesulfonate, e.g. p-hexyltoluenesulfonate (time period depending upon the length and bulkiness of the alkyl or derivatized alkyl). The reaction can be followed by TLC in a solvent system 80:10:10 (acetonitrile:KNO₃ (aq. saturated):H₂O), until single spot is obtained. (With longer chains the atropoisomers will emerge and multiple spots will be observed). Whether atropoisomers are resolved or incomplete quaternization occurs may be determined by mass spectrometry. The mixture may then be washed with chloroform and water in a separatory funnel to remove toluenesulfonate and DMF. The aqueous phase is used to isolate the chloride salt as described below. In an alternate method, the derivatization may be carried out with an alkyl (or derivatized alkyl) halide.

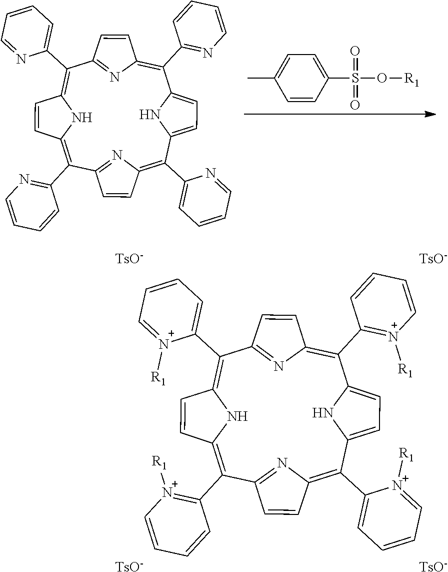

In the aqueous phase the porphyrin is precipitated first from water with NH₄ PF₆ as the PF₆⁻ salt, and subsequently washed extensively with diethylether. The PF₆⁻ salt can then be dissolved in acetone and then the chloride salt may be precipitated from acetone with tetrabutylammonium chloride and washed thoroughly with acetone.

In a third step the insertion of Mn is carried out in aqueous solution upon increasing pH to 12.3 with 20-fold excess MnCl₂. The completion can be monitored by UV/vis and by TLC (same solvent as above) (as the absence of the fluorescent spot of metal-free porphyrin). The excess of Mn (as hydroxo/oxo complexes) is removed by double filtration (over filter paper) and then the Mn porphyrin is precipitated first as the PF6 salt from water, (depicted below) and then as chloride salt from acetone as described above for the metal-free ligand. The precipitation is done twice to assure the full removal of the water-soluble low-molecular weight Mn complexes.

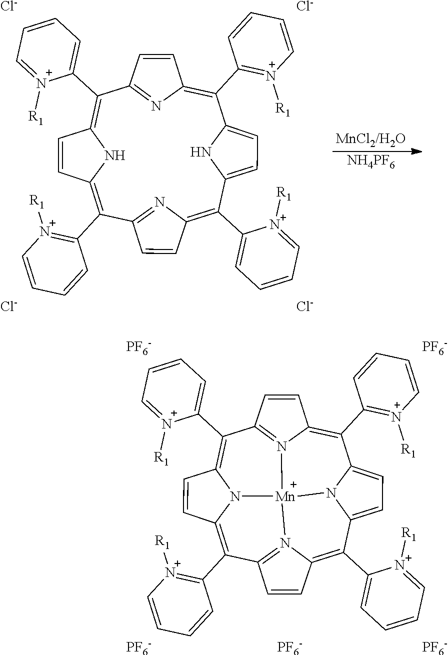

Further methods of synthesizing the substituted porphyrins of the formulae herein will be evident to those of ordinary skill in the art. Additionally, the various synthetic steps may be performed in an alternate sequence or order to give the desired compounds. Synthetic chemistry transformations useful in synthesizing the compounds described herein are known in the art and include, for example, those such as described in R. Larock, Comprehensive Organic Transformations, VCR Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

### Compositions Comprising Substituted Porphyrins

In one embodiment, the substituted porphyrins are administered in a pharmaceutically acceptable composition, such as in or with a pharmaceutically acceptable carrier or excipient. “Pharmaceutically acceptable carrier” means a carrier that is useful for the preparation of a pharmaceutical composition, i.e., generally compatible with the other ingredients of the composition. “A pharmaceutically acceptable carrier” includes both one and more than one carrier. Embodiments include carriers for topical, parenteral, intravenous, intraperitoneal intramuscular, sublingual, nasal and oral administration. “Pharmaceutically acceptable carrier” also includes agents for preparation of aqueous dispersions and sterile powders for injection or dispersions. “Excipient” as used herein includes compatible additives useful in preparation of a pharmaceutical composition. Examples of pharmaceutically acceptable carriers and excipients can for example be found in Remington Pharmaceutical Science, 16th Ed.

Compositions may include one or more of the isoforms of the substituted porphyrins of the present invention. When racemates exists, each enantiomer or diastereomer may be separately used, or they may be combined in any proportion. Where tautomers exist all possible tautomers are specifically contemplated.

Pharmaceutical compositions for use in accordance with the present invention may be formulated in a conventional manner using one or more pharmaceutically acceptable carriers or excipients. Thus, the substituted porphyrins may be formulated for administration by, for example, solid dosing, injection, implants, or oral, buccal, parenteral or rectal administration. Techniques and formulations may generally be found in “Remington's Pharmaceutical Sciences” (Meade Publishing Co., Easton, Pa.).

The route by which the substituted porphyrins of the present invention (component A) will be administered and the form of the composition will dictate the type of carrier (component B) to be used. The composition may be in a variety of forms, suitable, for example, for systemic administration (e.g., oral, rectal, nasal, sublingual, buccal, implants, or parenteral).

Carriers for systemic administration typically comprise at least one of a) diluents, b) lubricants, c) binders, d) disintegrants, e) colorants, f) flavors, g) sweeteners, h) antioxidants, j) preservatives, k) glidants, m) solvents, n) suspending agents, o) wetting agents, p) surfactants, combinations thereof, and others. All carriers are optional in the systemic compositions.

Ingredient a) is a diluent. Suitable diluents for solid dosage forms include sugars such as glucose, lactose, dextrose, and sucrose; diols such as propylene glycol; calcium carbonate; sodium carbonate; sugar alcohols, such as glycerin, mannitol, and sorbitol. The amount of ingredient a) in the systemic or topical composition is typically about 50 to about 90%.

Ingredient b) is a lubricant. Suitable lubricants for solid dosage forms are exemplified by solid lubricants including silica, talc, stearic acid and its magnesium salts and calcium salts, calcium sulfate; and liquid lubricants such as polyethylene glycol; and vegetable oils such as peanut oil, cottonseed oil, sesame oil, olive oil, corn oil and oil of theobroma. The amount of ingredient b) in the systemic or topical composition is typically about 5 to about 10%.

Ingredient c) is a binder. Suitable binders for solid dosage forms include polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The amount of ingredient c) in the systemic composition is typically about 5 to about 50%.

Ingredient d) is a disintegrant. Suitable disintegrants for solid dosage forms include agar, alginic acid and the sodium salt thereof, effervescent mixtures, croscarmelose, crospovidone, sodium carboxymethyl starch, sodium starch glycolate, clays, and ion exchange resins. The amount of ingredient d) in the systemic or topical composition is typically about 0.1 to about 10%.

Ingredient e) for solid dosage forms is a colorant such as an FD&C dye. When used, the amount of ingredient e) in the systemic or topical composition is typically about 0.005 to about 0.1%.

Ingredient f) for solid dosage forms is a flavor such as menthol, peppermint, and fruit flavors. The amount of ingredient f), when used, in the systemic or topical composition is typically about 0.1 to about 1.0%.

Ingredient g) for solid dosage forms is a sweetener such as aspartame and saccharin. The amount of ingredient g) in the systemic or topical composition is typically about 0.001 to about 1%.

Ingredient h) is an antioxidant such as butylated hydroxyanisole (“BHA”), butylated hydroxytoluene (“BHT”), and vitamin E. The amount of ingredient h) in the systemic or topical composition is typically about 0.1 to about 5%.

Ingredient j) is a preservative such as benzalkonium chloride, methyl paraben and sodium benzoate. The amount of ingredient j) in the systemic or topical composition is typically about 0.01 to about 5%.

Ingredient k) for solid dosage forms is a glidant such as silicon dioxide. The amount of ingredient k) in the systemic or topical composition is typically about 1 to about 5%.

Ingredient m) is a solvent, such as water, isotonic saline, ethyl oleate, glycerine, hydroxylated castor oils, alcohols such as ethanol, and phosphate buffer solutions. The amount of ingredient m) in the systemic or topical composition is typically from about 0 to about 100%.

Ingredient n) is a suspending agent. Suitable suspending agents include Avicel® RC-591 (from FMC Corporation of Philadelphia, Pa.) and sodium alginate. The amount of ingredient n) in the systemic or topical composition is typically about 1 to about 8%.

Ingredient o) is a surfactant such as lecithin, Polysorbate 80, and sodium lauryl sulfate, and the TWEENS® from Atlas Powder Company of Wilmington, Del. Suitable surfactants include those disclosed in the C.T.F.A. Cosmetic Ingredient Handbook, 1992, pp. 587-592; Remington's Pharmaceutical Sciences, 15th Ed. 1975, pp. 335-337; and McCutcheon's Volume 1, Emulsifiers & Detergents, 1994, North American Edition, pp. 236-239. The amount of ingredient o) in the systemic or topical composition is typically about 0.1% to about 5%.

Although the amounts of components A and B in the systemic compositions will vary depending on the type of systemic composition prepared, the specific derivative selected for component A and the ingredients of component B, in general, system compositions comprise about 0.01% to about 50% of component A and about 50% to about 99.99% of component B.

Compositions for parenteral administration typically comprise A) about 0.01 to about 10% of the substituted porphyrins of the present invention and B) about 90 to about 99.99% of a carrier comprising a) a diluent and m) a solvent. In one embodiment, component a) comprises propylene glycol and m) comprises ethanol or ethyl oleate.

Compositions for oral administration can have various dosage forms. For example, solid forms include tablets, capsules, granules, and bulk powders. These oral dosage forms comprise a safe and effective amount, usually at least about 5%, and more particularly from about 25% to about 50% of component A). The oral dosage compositions further comprise about 50 to about 95% of component B), and more particularly, from about 50 to about 75%.

Tablets can be compressed, tablet triturates, enteric-coated, sugar-coated, film-coated, or multiple-compressed. Tablets typically comprise component A, and component B a carrier comprising ingredients selected from the group consisting of a) diluents, b) lubricants, c) binders, d) disintegrants, e) colorants, f) flavors, g) sweeteners, k) glidants, and combinations thereof. Specific diluents include calcium carbonate, sodium carbonate, mannitol, lactose and cellulose. Specific binders include starch, gelatin, and sucrose. Specific disintegrants include alginic acid and croscarmelose. Specific lubricants include magnesium stearate, stearic acid, and talc. Specific colorants are the FD&C dyes, which can be added for appearance. Chewable tablets preferably contain g) sweeteners such as aspartame and saccharin, or f) flavors such as menthol, peppermint, fruit flavors, or a combination thereof.

Capsules (including implants, time release and sustained release formulations) typically comprise component A, and a carrier comprising one or more a) diluents disclosed above in a capsule comprising gelatin. Granules typically comprise component A, and preferably further comprise k) glidants such as silicon dioxide to improve flow characteristics. Implants can be of the biodegradable or the non-biodegradable type. Implants may be prepared using any known biocompatible formulation.

The selection of ingredients in the carrier for oral compositions depends on secondary considerations like taste, cost, and shelf stability, which are not critical for the purposes of this invention. One skilled in the art would know how to select appropriate ingredients without undue experimentation.

The solid compositions may also be coated by conventional methods, typically with pH or time-dependent coatings, such that component A is released in the gastrointestinal tract in the vicinity of the desired application, or at various points and times to extend the desired action. The coatings typically comprise one or more components selected from the group consisting of cellulose acetate phthalate, polyvinyl acetate phthalate, hydroxypropyl methyl cellulose phthalate, ethyl cellulose, EUDRAGIT® coatings (available from Rohm & Haas G.M.B.H. of Darmstadt, Germany), waxes and shellac.

Compositions for oral administration can also have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically comprise component A and component B, namely, a carrier comprising ingredients selected from the group consisting of a) diluents, e) colorants, f) flavors, g) sweeteners, j) preservatives, m) solvents, n) suspending agents, and o) surfactants. Peroral liquid compositions preferably comprise one or more ingredients selected from the group consisting of e) colorants, f) flavors, and g) sweeteners.

Other compositions useful for attaining systemic delivery of the subject substituted porphyrins include sublingual, buccal and nasal dosage forms. Such compositions typically comprise one or more of soluble filler substances such as a) diluents including sucrose, sorbitol and mannitol; and c) binders such as acacia, microcrystalline cellulose, carboxymethyl cellulose, and hydroxypropyl methylcellulose. Such compositions may further comprise b) lubricants, e) colorants, f) flavors, g) sweeteners, h) antioxidants, and k) glidants.

The amount of the carrier employed in conjunction with component A is sufficient to provide a practical quantity of composition for administration per unit dose of the medicament. Techniques and compositions for making dosage forms useful in the methods of this invention are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2nd Ed., (1976).

Component B may comprise a single ingredient or a combination of two or more ingredients.

Component A may be included in kits comprising component A, a systemic composition described above, or both; and information, instructions, or both that use of the kit will provide treatment for cosmetic and medical conditions in mammals (particularly humans). The information and instructions may be in the form of words, pictures, or both, and the like. In addition or in the alternative, the kit may comprise the medicament, a composition, or both; and information, instructions, or both, regarding methods of application of medicament, or of composition, preferably with the benefit of treating or preventing cosmetic and medical conditions in mammals (e.g., humans).

### Method of Using Substituted Porphyrins

Described herein are methods of treating ischemic injury, subarachnoid hemorrhage, spinal cord injury or traumatic brain injury comprising administering to a subject in need thereof a therapeutically effective amount of a substituted porphyrin. Also described are methods of providing neuroprotection comprising administering to a subject in need thereof a therapeutically effective amount of a substituted porphyrin.

In one aspect, the invention may provide a method of treating ischemic injury comprising administering a therapeutically effective amount of a substituted porphyrin to a subject in need thereof more than 4.5 hours post ischemia onset. In another aspect, the invention may provide a method of treating ischemic injury comprising administering a therapeutically effective amount of a substituted porphyrin to a subject in need thereof more than 6 hours post ischemia onset. In another aspect, the invention may provide a method of treating ischemic injury comprising administering a therapeutically effective amount of a substituted porphyrin to a subject in need thereof at least once per day for at least 5 days post ischemia onset.

In some embodiments, the ischemic injury may be cerebral ischemia or stroke or spinal cord ischemia or traumatic brain injury. The substituted porphyrins may be administered more than about 4.5 hours post ischemia onset, more than 6 hours post ischemia onset, more than about 8 hours post ischemia onset or more than about 10 hours post ischemia onset. Alternatively, in other embodiments, the substituted porphyrins may be administered more than about 4.5 hours post reperfusion.

The substituted porphyrins may be administered for about 1 week or about 2 weeks or about 3 weeks or about 4 weeks post ischemia onset. The substituted porphyrins may be administered daily, twice a day, three times daily or four times daily. Alternatively, in another embodiment, the substituted porphyrins may be administered continuously, such as via intravenous administration. In other embodiments, the substituted porphyrins may be administered once weekly or twice weekly.

In another aspect, the present invention may provide a method of providing neuroprotection comprising administering a therapeutically effective amount of a substituted porphyrin to a subject in need thereof more than 4.5 hours post ischemia onset. In another aspect, the present invention may provide a method of providing neuroprotection comprising administering a therapeutically effective amount of a substituted porphyrin to a subject in need thereof more than 6 hours post ischemia onset. In another aspect, the present invention may provide a method of providing neuroprotection comprising administering a therapeutically effective amount of a substituted porphyrin to a subject in need thereof at least once per day for at least 5 days post ischemia onset.

As used herein, the term “neuroprotection” includes protecting a neuron as well as resuscitating a neuron (“neuroresuscitation”). The substituted porphyrins may be administered more than about 4.5 hours post ischemia onset, more than about 6 hours post ischemia onset, more than about 8 hours post ischemia onset or more than about 10 hours post ischemia onset. Alternatively, in other embodiments, the substituted porphyrins may be administered more than about 4.5 hours post reperfusion.

The substituted porphyrins may be administered for about 1 week or about 2 weeks or about 3 weeks or about 4 weeks post ischemia onset. The substituted porphyrins may be administered daily, twice a day, three times daily or four times daily. Alternatively, in another embodiment, the substituted porphyrins may be administered continuously, such as via intravenous administration. In other embodiments, the substituted porphyrins may be administered once weekly or twice weekly.

In yet another embodiment, the present invention provides a method of treating subarachnoid hemorrhage comprising administering a therapeutically effective amount of a substituted porphyrin to a subject in need thereof.

In some embodiments, the substituted porphyrin may be administered at more than about 4.5 hours post hemorrhage, more than about 6 hours post hemorrhage, more than about 8 hours post hemorrhage or more than about 10 hours post hemorrhage. In some embodiments, the substituted porphyrin may be administered to the subject in need thereof at least once per day for at least 5 days post hemorrhage.

The substituted porphyrins may be administered for about 1 week or about 2 weeks or about 3 weeks or about 4 weeks post hemorrhage. The substituted porphyrins may be administered daily, twice a day, three times daily or four times daily. Alternatively, in another embodiment, the substituted porphyrins may be administered continuously, such as via intravenous administration. In other embodiments, the substituted porphyrins may be administered once weekly or twice weekly.

In another aspect, the present invention may provide a method of treating traumatic brain injury (TBI) comprising administering a therapeutically effective amount of a substituted porphyrin to a subject in need thereof.

In some embodiments, the substituted porphyrin may be administered at more than about 4.5 hours post TBI, more than about 6 hours post TBI, more than about 8 hours post TBI or more than about 10 hours post TBI. In some embodiments, the substituted porphyrin may be administered to the subject in need thereof at least once per day for at least 5 days post TBI.

The substituted porphyrins may be administered for about 1 week or about 2 weeks or about 3 weeks or about 4 weeks post TBI. The substituted porphyrins may be administered daily, twice a day, three times daily or four times daily. Alternatively, in another embodiment, the substituted porphyrins may be administered continuously, such as via intravenous administration. In other embodiments, the substituted porphyrins may be administered once weekly or twice weekly.

In another aspect, the present invention may provide a method of treating spinal cord injury (SCI) comprising administering a therapeutically effective amount of a substituted porphyrin to a subject in need thereof.

In some embodiments, the substituted porphyrin may be administered at more than about 4.5 hours post SCI, more than about 6 hours post SCI, more than about 8 hours post SCI or more than about 10 hours post SCI. In some embodiments, the substituted porphyrin may be administered to the subject in need thereof at least once per day for at least 5 days post SCI.

The substituted porphyrins may be administered for about 1 week or about 2 weeks or about 3 weeks or about 4 weeks post SCI. The substituted porphyrins may be administered daily, twice a day, three times daily or four times daily. Alternatively, in another embodiment, the substituted porphyrins may be administered continuously, such as via intravenous administration. In other embodiments, the substituted porphyrins may be administered once weekly or twice weekly.

As used herein, “subject” may be a eukaryote, an animal, a vertebrate animal, a mammal, a rodent (e.g. a guinea pig, a hamster, a rat, a mouse), murine (e.g. a mouse), canine (e.g. a dog), feline (e.g. a cat), equine (e.g. a horse), a primate, simian (e.g. a monkey or ape), a monkey (e.g. marmoset, baboon), an ape (e.g. gorilla, chimpanzee, orangutan, gibbon), or a human.

The term “treatment”, as used herein in the context of treating a condition, pertains generally to treatment and therapy, whether of a human or an animal (e.g. in veterinary applications), in which a desired therapeutic effect is achieved. For example, treatment may ameliorate the condition or may inhibit the progress of the condition (e.g., reduce the rate of progress or halt the rate of progress).

A therapeutically effective amount of a substituted porphyrin according to the present invention will vary with the particular condition being treated, the age and physical condition of the subject being treated, the severity of the condition, the duration of treatment, the nature of concurrent therapy, the route of administration, the particular pharmaceutically-acceptable carrier utilized, and like factors within the knowledge and expertise of the attending physician. For example, an effective amount of the substituted porphyrins of the present invention for systemic administration is from about 0.01 to about 100 mg/kg body weight, preferably from about 0.1 to about 100 mg/kg per body weight, most preferably from about 1 to about 50 mg/kg body weight per day. Plasma levels for systemic administration are expected to be in the range of 0.001 to 100 microgram/mL, more preferably from 0.01 to 50 microgram/mL and most preferably from 0.1 to 10 microgram/mL. While these dosages are based upon a daily administration rate, the substituted porphyrins of the present invention may also be administered at other intervals, such as twice per day, twice weekly, once weekly, or once a month. The substituted porphyrins of the present invention may also be administered in a continuous mode, for example, using a pump. In one embodiment, the porphyrins may be initially administered more frequently (e.g. daily) at higher doses to establish a loading dose with continued administration at a lower less frequent dose. One of ordinary skill in the art would be able to calculate suitable effective amounts for other intervals of administration. For example, the efficacy of various substituted porphyrins in vivo is affected by both the potency of the substituted porphyrin and the bioavailability of that porphyrin.

In some embodiments, an additional active agent or agents can be administered with the substituted porphyrins in the methods of the present invention. The additional active agent or agents can be administered simultaneously or sequentially with the substituted porphyrins of the present invention. Sequential administration includes administration before or after the substituted porphyrins of the present invention. In some embodiments, the additional active agent or agents can be administered in the same composition as the substituted porphyrins of the present invention. In other embodiments, there can be an interval of time between administration of the additional active agent and the substituted porphyrins of the present invention.

In some embodiments, the administration of an additional therapeutic agent with a compound of the present invention will enable lower doses of the other therapeutic agents to be administered for a longer period of time.

### Ischemic Injuries

Ischemia refers to a reduction or abolition of blood supply to a tissue. The methods described herein can be used to treat injuries associated with ischemia, or “ischemic injuries.” Ischemic injuries can include injuries to, e.g., the kidney, liver, lungs, pancreas, skeletal muscle, intestines, heart and brain. Ischemic injuries can be associated with or caused by, e.g., acute myocardial infarction, elective angioplasty, coronary artery bypass graft, surgery involving cardiac bypass or organ or tissue transplantation (e.g., cardiac transplantation), tissue rejection after transplantation, graft versus host disease, stroke, head trauma, drowning, sepsis, cardiac arrest, shock, atherosclerosis, hypertension, cocaine-induced heart disease, smoking-induced heart disease, heart failure, pulmonary hypertension, hemorrhage, capillary leak syndrome (such as child and adult respiratory distress syndrome), multi-organ system failure, a state of low colloid oncotic pressure (such as starvation, anorexia nervosa, or hepatic failure with decreased production of serum proteins), anaphylaxis, hypothermia, cold injury (e.g., due to hypothermic perfusion or frostbite) hepatorenal syndrome, delirium tremens, a crush injury, mesenteric insufficiency, peripheral vascular disease, claudication, burn, electrocution, excessive drug-induced vasodilation, excessive drug-induced vasoconstriction, radiation exposure (e.g., during fluoroscopy or radiographic imaging), or exposure to high energy, e.g., exposure to laser light. Excessive drug-induced vasodilation can be caused by, for instance, nitroprusside, hydralazone, dyazoxide, a calcium channel blocker, or a general anesthetic. Excessive drug-induced vasoconstriction can be caused by, for instance, neosynephrine, isoproterenol, dopamine, dobutamine, or cocaine.

### Ischemia-Reperfusion Injury

“Ischemia-reperfusion injury” refers to an injury resulting from the reestablishment (reperfusion) of the flow of blood to a region of the body following a temporary halt in the flow. For example, ischemia-reperfusion injury can occur during certain surgical procedures, such as repair of aortic aneurysms and organ transplantation. Clinically, ischemia-reperfusion injury can be manifested by complications such as, e.g., pulmonary dysfunction, including adult respiratory distress syndrome, renal dysfunction, consumptive coagulopathies including thrombocytopenia, fibrin deposition into the microvasculature and disseminated intravascular coagulopathy, transient and permanent spinal cord injury, cardiac arrhythmias and acute ischemic events, hepatic dysfunction including acute hepatocellular damage and necrosis, gastrointestinal dysfunction including hemorrhage and/or infarction and multisystem organ dysfunction (MSOD) or acute systemic inflammatory distress syndromes (SIRS). The injury may occur in the parts of the body to which the blood supply was interrupted, or it can occur in parts fully supplied with blood during the period of ischemia.

### Stroke

Stroke is a general term for acute brain damage resulting from disease or injury of blood vessels. Stroke can be classified into at least two main categories: hemorrhagic stroke (resulting from leakage of blood outside of the normal blood vessels) and ischemic stroke (cerebral ischemia due to lack of blood supply). Some events that can cause ischemic stroke include thrombosis, embolism, and systemic hypoperfusion (with resultant ischemia and hypoxia).

Stroke generally causes neuronal death and injury in the brain by oxygen deprivation and secondary events. The area of the brain that dies as a result of the lack of blood supply or other damage is called an infarct. In some cases, the treatments described herein can be used to reduce or minimize the size of an infarct, e.g., by reducing secondary events that cause neuronal death or injury.

Obstruction of a cerebral artery resulting from a thrombus which has built up on the wall of a brain artery is generally called cerebral thrombosis. In cerebral embolism, the occlusive material blocking the cerebral artery arises downstream in the circulation (e.g., an embolus is carried to the cerebral artery from the heart). Because it is difficult to discern whether a stroke is caused by thrombosis or embolism, the term thromboembolism is used to cover both these types of stroke. Systemic hypoperfusion may arise as a consequence of decreased blood levels, reduced hematocrit, low blood pressure or inability of the heart to pump blood adequately.

Thrombolytic agents, such as tissue plasminogen activator (t-PA), have been used in the treatment of thromboembolic stroke. These molecules function by lysing the thrombus causing the ischemia. Such drugs are believed to be most useful if administered as soon as possible after acute stroke (preferably within 3 hours) in order to at least partially restore cerebral blood flow in the ischemic region and to sustain neuronal viability. A substituted porphyrin can be used, instead of or in combination with, such thrombolytic agents, to achieve a therapeutic benefit in a subject who has experienced a thromboembolic stroke.

### Subarachnoid Hemorrhage

Subarachnoid hemorrhage (SAH) constitutes sudden bleeding (extravasation of blood) into the subarachnoid space of the central nervous system. SAH is classified as spontaneous or traumatic. Spontaneous SAH usually results from a ruptured intracranial aneurysm. Traumatic SAH usually results from a bicycle, motorcycle or automobile accident or accidental fall or a sports related cause.

Symptoms of subarachnoid hemorrhage include acute severe headache, vomiting, dizziness, loss of consciousness, coma, stiff neck, fever, aversion to light and neurologic deficits, e.g., partial paralysis, loss of vision, seizures and speech difficulties.

### Other Stroke Treatments

A stroke treatment can involve the use of one or more substituted porphyrins that can be used in combination with one or more stroke treatments. The treatments can be administered at the same time, but also at separate times, e.g., at separate times that are within a specified interval, e.g., within the same 48, 24, 12, 6, 2, or 1 hour. Furthermore, the treatments can be using distinct modes of administration.

Treatments that can be administered in combination with a substituted porphyrin include: a thrombolytic agent (e.g., streptokinase, acylated plasminogen-streptokinase activator complex (APSAC), urokinase, single-chain urokinase-plasminogen activator (scu-PA), other anti-inflammatory agents, thrombin-like enzymes from snake venoms such as ancrod, thrombin inhibitors, tissue plasminogen activator (t-PA) and biologically active variants of each of the above); an anticoagulant (e.g., warfarin or heparin); antiplatelet drug (e.g., aspirin); a glycoprotein IIb/IIIa inhibitor; a glycosaminoglycan; coumarin; GCSF; melatonin; a caspase inhibitor; an anti-oxidants (e.g., NXY-059, see Lees et al., (2006) N. Engl. J. Med 354, 588-600), a neuroprotectant (e.g., an NMDA receptor antagonist and a cannabinoid antagonist), an anti-CD 18 antibody; an anti-CDI Ia antibody; an anti-ICAM-1 antibody; an anti-VLA-4 antibody, an anti-TWEAK antibody, an anti-TWEAK-R antibody, carotid endarterectomy; angioplasty; insertion of a stent; and an alternative medicine (e.g., acupuncture, traditional Chinese medicine, meditation, massage, hyperbaric oxygen treatment, or conductive pedagogy).

### Stroke Assessment Criteria

The ability of a substituted porphyrin to treat a subject can be evaluated, subjectively or objectively, e.g., using a variety of criteria. A number of assessment tools are available to provide the evaluation.

Exemplary prehospital stroke assessment tools include the Cincinnati Stroke Scale and the Los Angeles Prehospital Stroke Screen (LAPSS). Acute assessment scales include, e.g., the Canadian Neurological Scale (CNS), the Glasgow Coma Scale (GCS), the Hempispheric Stroke Scale, the Hunt & Hess Scale, the Mathew Stroke Scale, the Mini-Mental State Examination (MMSE), the NIH Stroke Scale (NIHSS), the Orgogozo Stroke Scale, the Oxfordshire Community Stroke Project Classification (Bamford), and the Scandinavian Stroke Scale. Functional assessment scales include the Berg Balance Scale, the Modified Rankin Scale, the Stroke Impact Scale (SIS), and the Stroke Specific Quality of Life Measure (SS-QOL). Outcome assessment tools include the American Heart Association Stroke Outcome Classification (AHA SOC), the Barthel Index, the Functional Independence Measurement (FIM™), the Glasgow Outcome Scale (GOS), and the Health Survey SF-36™ & SF-12™. Other diagnostic and screening tests include the Action Research Arm Test, the Blessed-Dementia Scale, the Blessed-Dementia Information-Memory-Concentration Test, the DSM-IV criteria for the diagnosis of vascular dementia, the Hachinkski Ischaemia Score, the Hamilton Rating Scale for Depression, the NINDS-AIREN criteria for the diagnosis of vascular dementia, the Orpington Prognostic Score, the Short Orientation-Memory-Concentration Test, the Thrombosis In Myocardial Infarction grading scheme, MRI imaging (e.g., diffusion and perfusion imaging techniques (Henninger et al., Stroke 37: 1283-1287, 2006), diffusion-weighted (DWI) MRI techniques, and flow-sensitive imaging, e.g., fluid-attenuated inversion recovery (FLAIR)), functional and spectroscopical imaging (Koroshetz, Ann. Neural. 39:283-284, 1996), and PET (Heiss et al., Cerebrovasc. Brain Metab. Rev. 5:235-263, 1993), and.

An evaluation can be performed before and/or after the administration of a substituted porphyrin.

### Traumatic Brain Injury

A substituted porphyrin can be used to treat traumatic brain injury. Damage to the brain by a physical force is broadly termed traumatic brain injury (TBI). The resulting effect of TBI causes alteration of normal brain processes attributable to changes in brain structure and/or function. There are two basic types of brain injury, open head injury and closed head injury. In an open head injury, an object, such as a bullet, penetrates the skull and damages the brain tissue. Closed head injury is usually caused by a rapid movement of the head during which the brain is whipped back and forth, bouncing off the inside of the skull. Closed head injuries are the more common of the two, which often result from accidents involving motor vehicles or falls. In a closed head injury, brute force or forceful shaking injures the brain. The stress of this rapid movement pulls apart and stretches nerve fibers or axons, breaking connections between different parts of the brain. In most cases, a resulting blood clot, or hematoma, may push on the brain or around it, raising the pressure inside the head. Both open and closed head injuries can cause severe damage to the brain, resulting in the need for immediate medical attention.

Depending on the type of force that hits the head, varying injuries such as any of the following can result: jarring of the brain within the skull, concussion, skull fracture, contusion, subdural hematoma, or diffuse axonal injury. Though each person's experience is different, there are common problems that many people with TBI face. Possibilities documented include difficulty in concentrating, ineffective problem solving, short and long-term memory problems, and impaired motor or sensory skills; to the point of an inability to perform daily living skills independently such as eating, dressing or bathing. The most widely accepted concept of brain injury divides the process into primary and secondary events. Primary brain injury is considered to be more or less complete at the time of impact, while secondary injury evolves over a period of hours to days following trauma.

Primary injuries are those commonly associated with emergency situations such as auto accidents, or anything causing temporary loss of consciousness or fracturing of the skull. Contusions, or bruise-like injuries, often occur under the location of a particular impact. The shifting and rotating of the brain inside the skull after a closed brain injury results in shearing injury to the brain's long connecting nerve fibers or axons, which is referred to as diffuse axonal injury. Lacerations are defined as the tearing of frontal and temporal lobes or blood vessels caused by the brain rotating across ridges inside the skull. Hematomas, or blood clots, result when small vessels are broken by the injury. They can occur between the skull and the brain (epidural or subdural hematoma), or inside the substance of the brain itself (intracerebral hematoma). In either case, if they are sufficiently large they will compress or shift the brain, damaging sensitive structures within the brain stem. They can also raise the pressure inside the skull and eventually shut off the blood supply to the brain.

Delayed secondary injury at the cellular level has come to be recognized as a major contributor to the ultimate tissue loss that occurs after brain injury. A cascade of physiologic, vascular, and biochemical events is set in motion in injured tissue. This process involves a multitude of systems, including possible changes in neuropeptides, electrolytes such as calcium and magnesium, excitatory amino acids, arachidonic acid metabolites such as the prostaglandins and leukotrienes, and the formation of oxygen free radicals. This secondary tissue damage is at the root of most of the severe, long-term adverse effects a person with brain injury may experience. Procedures that minimize this damage can be the difference between recovery to a normal or near-normal condition, or permanent disability.

Diffuse blood vessel damage has been increasingly implicated as a major component of brain injury. The vascular response seems to be biphasic. Depending on the severity of the trauma, early changes include an initial rise in blood pressure, an early loss of the automatic regulation of cerebral blood vessels, and a transient breakdown of the blood-brain barrier (BBB). Vascular changes peak at approximately six hours post-injury but can persist for as long as six days. The clinical significance of these blood vessels changes is still unclear, but may relate to delayed brain swelling that is often seen, especially in younger people. The process by which brain contusions produce brain necrosis is equally complex and is also prolonged over a period of hours. Toxic processes include the release of oxygen free radicals, damage to cell membranes, opening of ion channels to an influx of calcium, release of cytokines, and metabolism of free fatty acids into highly reactive substances that may cause vascular spasm and ischemia. Free radicals are formed at some point in almost every mechanism of secondary injury. The primary target of the free radicals is fatty acids of the cell membrane. A process known as lipid peroxidation damages neuronal, glial, and vascular cell membranes in a geometrically progressing fashion. If unchecked, lipid peroxidation spreads over the surface of the cell membrane and eventually leads to cell death. Thus, free radicals damage endothelial cells, disrupt the blood-brain barrier (BBB), and directly injure brain cells, causing edema and structural changes in neurons and glia. Disruption of the BBB is responsible for brain edema and exposure of brain cells to damaging blood-borne products.

Secondary systemic insults (outside the brain) may consequently lead to further damage to the brain. This is extremely common after brain injuries of all grades of severity, particularly if they are associated with multiple injuries. Thus, people with brain injury may experience combinations of low blood oxygen, blood pressure, heart and lung changes, fever, blood coagulation disorders, and other adverse changes at recurrent intervals in the days following brain injury. These occur at a time when the normal regulatory mechanism, by which the cerebral blood vessels can relax to maintain an adequate supply of oxygen and blood during such adverse events, is impaired as a result of the original trauma. The protocols for immediate assessment are limited in their efficiency and reliability and are often invasive. Computer-assisted tomographic (CT) scanning is currently accepted as the standard diagnostic procedure for evaluating TBI, as it can identify many abnormalities associated with primary brain injury, is widely available, and can be performed at a relatively low cost (Marik et al. Chest 122:688-711 2002; McAllister et al. Journal of Clinical and Experimental Neuropsychology 23:775-791 2001). However, the use of CT scanning in the diagnosis and management of patients presenting to emergency departments with TBI can vary among institutions, and CT scan results themselves may be poor predictors of neuropsychiatric outcome in TBI subjects, especially in the case of mild TBI injury (McCullagh et al. Brain Injury 15:489-497 2001).

Immediate treatment for TBI typically involves surgery to control bleeding in and around the brain, monitoring and controlling intracranial pressure, insuring adequate blood flow to the brain, and treating the body for other injuries and infection. Those with mild brain injuries often experience subtle symptoms and may defer treatment for days or even weeks. Once a patient chooses to seek medical attention, observation, neurological testing, magnetic resonance imaging (MRI), positron emission tomography (PET) scan, single-photon emission CT (SPECT) scan, monitoring the level of a neurotransmitter in spinal fluid, computed tomography (CT) scans, and X-rays may be used to determine the extent of the patient's injury. The type and severity of the injury determine further care.

A substituted porphyrin can be used, alone or in combination with another treatment, to achieve a therapeutic benefit in a subject who has experienced a TBI. For example, a substituted porphyrin can be used to treat a primary injury, a secondary injury, or both. Alternatively, a substituted porphyrin can be used to treat a primary injury and as a prophylactic therapy for a secondary injury. An evaluation can be performed before and/or after the administration of a substituted porphyrin.

### Spinal Cord Injury

A substituted porphyrin can also be used to treat spinal cord injury. Spinal cord injury (SCI) is an insult to the spinal cord resulting in a change, either temporary or permanent, in its normal motor, sensory, or autonomic function. Both clinical and experimental studies evidence that the spinal cord suffers from primary and secondary damage after acute SCI. Primary SCI arises from mechanical disruption, transection, extradural pathology, or distraction of neural elements. This injury usually occurs with fracture and/or dislocation of the spine. However, primary SCI may occur in the absence of spinal fracture or dislocation. Penetrating injuries due to bullets or weapons may also cause primary SCI (Burney et al., Arch Surg 128(5): 596-9 (1993)). More commonly, displaced bone fragments cause penetrating spinal cord or segmental spinal nerve injuries. Extradural pathology may also cause primary SCI. Spinal epidural hematomas or abscesses cause acute cord compression and injury. Spinal cord compression from metastatic disease is a common oncologic emergency. Longitudinal distraction with or without flexion and/or extension of the vertebral column may result in primary SCI without spinal fracture or dislocation. A substituted porphyrin can be used to treat a primary spinal injury. The pathophysiology of secondary SCI involves a multitude of cellular and molecular events that progress over the first few days after injury (Tator, Brain Pathology 5:407-413 (1995)). The most important cause of secondary SCI is vascular injury to the spinal cord caused by arterial disruption, arterial thrombosis, and hypoperfusion due to shock. SCI can be sustained through ischemia from damage or impingement on the spinal arteries. SCI due to ischemia can occur during surgery where aortic blood flow is temporarily stopped. A substituted porphyrin can be used to treat or prevent secondary SCI injury. Spinal cord injury can also be caused by toxicity (Tator, Brain Pathology 5:407-413 (1995)). One of the most compelling toxicity in spinal cord injury is the accumulation and subsequent damage exerted by the excitatory amino acid neurotransmitter. Glutamate induced excitotoxicity causes an elevation of intracellular calcium. Raised intracellular calcium can in turn cause activation of calcium dependent proteases or lipases which cause further damage due to breakdown of cytoskeletal components including neurofilaments and dissolution of cell membranes. The excess production of arachidonic acid and eicosanoids such as prostaglandins may be related to lipid peroxidation and oxygen free radicals. The release of vasoactive eicosanoids from damaged neuronal membranes may in turn cause progressive posttraumatic ischemia by inducing vasospasm. Endogenous opioids may also be involved in the secondary injury process either by their effects on the local or systemic circulation or by direct effects on the injured cord. A substituted porphyrin can be used to treat or prevent spinal cord injury resulting from toxicity.

Significant and progressive edema can follow spinal cord injury. It is not known whether the edema is injurious in itself or whether it is an epiphenomenon of another injury mechanism such as ischemia or glutamate toxicity. Edema can spread in the cord from the site of injury for a considerable distance rostrally and caudally in both experimental models and clinical cases. Edema can cause increased spinal cord tissue pressure and a delayed secondary ischemic insult.

SCI are classified as complete or incomplete, based on the extent of injury, according to the American Spinal Injury Association (ASIA) Impairment Scale. In complete SCI, there is no sensory and motor function preserved in the lowest sacral segments (Waters et al, Paraplegia 29(9): 573-81 (1991)). In incomplete SCI, sensory or motor function is preserved below the level of injury including the lowest sacral segments (Waters et al., Archives of Physical Medicine and Rehabilitation 75(3): 306-11 (1994)). Incomplete cord lesions may evolve into more complete lesions. More commonly, the injury level rises one or two spinal levels during the hours to days after the initial event.

Other classifications of SCI include central cord syndrome, Brown-Sequard syndrome, anterior cord syndrome, conus medullaris syndrome and cauda equina syndrome. Central cord syndrome is often associated with a cervical region injury leading to greater weakness in the upper limbs than in the lower limbs with sacral sensory sparing. Brown-Sequard syndrome involves a hemisection lesion of the cord, causing a relatively greater ipsilateral proprioceptive and motor loss with contralateral loss of sensitivity to pain and temperature. Anterior cord syndrome is often associated with a lesion causing variable loss of motor function and sensitivity to pain and temperature, while proprioception is preserved. Conus medullaris syndrome is associated with injury to the sacral cord and lumbar nerve roots. This syndrome is characterized by areflexia in the bladder, bowel, and lower limbs, while the sacral segments occasionally may show preserved reflexes (e.g., bulbocavernosus and micturition reflexes). Cauda equina syndrome is due to injury to the lumbosacral nerve roots in the spinal canal, leading to areflexic bladder, bowel, and lower limbs. Neurogenic shock can result from SCI (Tator, Brain Pathology 5:407-413 (1995)). Neurogenic shock refers to the hemodynamic triad of hypotension, bradycardia, and peripheral vasodilation resulting from autonomic dysfunction and the interruption of sympathetic nervous system control in acute SCI, and is differentiated from spinal and hypovolemic shock. Hypovolemic shock tends to be associated with tachycardia. Spinal shock is defined as the complete loss of all neurologic function, including reflexes and rectal tone, below a specific level that is associated with autonomic dysfunction. An initial increase in blood pressure is noted due to the release of catecholamines, followed by hypotension. Flaccid paralysis, including of the bowel and bladder, is observed, and sometimes sustained priapism develops. These symptoms tend to last several hours to days until the reflex arcs below the level of the injury begin to function again.

Current therapy for SCI aims to improve motor function and sensation in patients with the disorder. Corticosteroids are the mainstay of therapy. Glucocorticoids such as methylprednisolone are thought to reduce the secondary effects of acute SCI, and the use of high-dose methylprednisolone in nonpenetrating acute SCI has become the standard of care in North America.

A substituted porphyrin can be used to treat any classification of SCI, or a symptom thereof, as described herein. A substituted porphyrin can be used alone or in combination with another known therapy for SCI.

### EXAMPLES

In the following examples, MnTnHex-2-PyP⁵⁺ refers to Mn(III) 5,10,15,20-tetrakis(N-n-hexylpyridinium-2-yl)porphyrin.

The following methods were used in the Examples unless stated otherwise.

### Surgical Preparation

Male Wistar rats (250-275 gm; Harlan Sprague Dawley, Inc. Indianapolis, Ind.) were anesthetized with 64-mg/kg intraperitoneal sodium pentobarbital and positioned in a stereotactic head frame. The skin was infiltrated with 1.0% lidocaine and a midline scalp incision was made. A burr hole was drilled over the left hemisphere, 7.2 mm anterior to the interauralline and 1.4 mm lateral to the sagittal suture. An intracerebroventricular (ICV) cannula (33 gauge) was positioned with the tip in the left lateral ventricle and fixed in place with screws and cyanoacrylate. The incision was closed with suture around this assembly. After emergence from anesthesia, the animals were returned to their cages with free access to water and food.

Following 2-3 days recovery, rats were allowed access to water but fasted from food for 12 hours to standardize glycemic state. Rats were then anesthetized with isoflurane in O₂. Following tracheal intubation, the lungs were mechanically ventilated to maintain normocapnia. A 22-g needle thermistor was percutaneously placed adjacent to the skull beneath the temporalis. Pericranial temperature was servoregulated at 37.5±0.1° C. by surface heating or cooling. The inspired isoflurane concentration was adjusted to 1.0-1.5% in 50% O₂/balance N₂. The tail artery was cannulated. The animals were then prepared for MCAO as previously described [Mackensen et al. J. Neurosci. 21:4582-4592, 2001; Longa et al. Stroke 20:84-91, 1989]. A midline cervical incision was made and the right common carotid artery was identified. The external carotid artery (ECA) was isolated and the occipital, superior thyroid, and external maxillary arteries were ligated and divided. The internal carotid artery (ICA) was dissected distally until the origin of the pterygopalatine artery was visualized. Following surgical preparation, a 20 min interval was allowed for physiologic stabilization.

Five min before MCAO onset, heparin (50 IU intra-arterial) was given to prevent intra-arterial thrombosis. A 0.25-mm diameter nylon monofilament, prepared with a silicone tip, was inserted into the ECA stump and passed distally through the ICA (20 mm from carotid bifurcation) until resistance was felt and the filament was secured. At MCAO onset, isoflurane was reduced to 0.8-1.0%.

After 90 min of MCAO, the occlusive filament was removed. The anesthetic state and pericranial temperature regulation were continued for an additional 100 min. The tail artery catheter was removed and the wounds were closed with suture. Isoflurane was discontinued. Upon recovery of the righting reflex, the tracheas were extubated and the animals were placed in an O₂ enriched environment (FIO₂=50%) for 1 hour. Animals were then randomized to experimental groups (see below).

### Neurologic Evaluation

At completion of the predefined recovery interval, rats underwent a neurologic examination to evaluate sensorimotor function. The neurological scoring system evaluates four different functions (general status, simple motor deficit, complex motor deficit, and sensory deficit). The score given to each animal (by an observer blinded to group assignment) was the sum of all four individual scores, 0 being the minimum (best) score and 48 being the maximum (worst) score. This examination was developed combining features from several neurologic evaluations reported for rat MCAO and has been used in two long-term MCAO outcome studies to assess for differences in treatment outcome (Yokoo et al. Anesth Analg. 2004; 99:896-903; Sakai et al. Anesthesiology. 2007; 106:92-99; discussion 98-10.) Values from this scoring system correlate well with total infarct volume in rats allowed to survive 8 weeks post-MCAO (R2 value of 0.78, P=0.006, Sakai et al.) as well as 2 and 8 weeks post-MCAO (Yokoo et al.). Details of the examination are provided in Table 2.

TABLE 2
Method for neurological analysis

| | | POINTS | | | | |
|---|---|---|---|---|---|---|
| | TEST | 0 | 1 | 2 | 3 | 4 |
| GENERAL | Spontaneous | Normal | Calm, quiet, | Somnolent, | Stuporous, some | No spontaneous |
| STATUS | Activity (5 min) | | Explores slowly | Minimal exploration | movements in place | movement |
| | Body | Normal | Slight asymmetry | Moderate | Prominent asymmetry | Extreme asymmetry |
| | Symmetry | | | asymmetry | | |
| | Gait (open | Normal | Stiff, inflexible | Limping | Trembling, drifting, | Does not walk |
| | bench top) | | | | falling | |
| SIMPLE | Forelimb | Normal | Light asymmetry | Marked asymmetry | Prominent asymmetry | No body/limb |
| MOTOR | Symmetry | | | | | movement |
| | Circling/bench | Not present | Predominantly | Circles to one side | Circles constantly | Pivoting, swaying, |
| | top | | one-sided turns | | to one side | or no movement |
| | Circling/holding | Not present | Tendency to turn to | Circles to one side | Pivots to one side | Does not advance |
| | tail | | one side | | sluggishly | |
| | Hind limb | Normal | Slow placement | No placement | — | — |
| | Placement | | | | | |
| COMPLEX | Vertical Screen | Normal | Climbs with strain, | Holds onto screen, | Slides down screen, | Slides immediately, |
| MOTOR | Climbing | | limb weakness | does not slip or | unsuccessful effort to | no effort to prevent |
| | | | present | climb | prevent fall | fall |
| | Beam Walking | Walks to | Walks to the middle | No walking, stays | No walking, stays less | Falls immediately |
| | (sec) | other end | of beam | more than 10 sec | than 10 sec | |
| SENSORY | Forelimb Touch | Normal | Withdraws slowly | No withdrawal | — | — |
| | (needle) | | | | | |
| | Hind Limb | Normal | Withdraws slowly | No withdrawal | — | — |
| | Touch (needle) | | | | | |
| | Trunk Touch | Symmetrical | Light asymmetry | Prominent | Absent ipsilateral and | Response absent |
| | (needle) | response | | asymmetry | diminished contralateral | bilaterally |
| | | | | | response | |
| | Vibrissae Touch | Symmetrical | Light asymmetry | Prominent | Absent response | Response absent |
| | | response | | asymmetry | ipsilaterally, diminished | bilaterally |
| | | | | | contralaterally | |
| | Face Touch | Normal | Withdraws slowly | No withdrawal | — | — |
| | (needle) | | | | | |

### Measurement of Cerebral Infarct Volume

Animals were weighed, anesthetized with isoflurane, and decapitated. The brains were removed, frozen at −40° C. in 2-methylbutane, and stored at −70° C. Infarct volume was measured by comparing the volume of histologically normal tissue observed in the ischemic hemisphere to the expected volume of normal tissue as derived from measurements of the contralateral, non-ischemic hemisphere [Swanson et al. J. Cereb. Blood Flow Metab. 10:290-293, 1990]. Serial quadruplicate 20-mm thick coronal sections were taken using a cryotome at 660-mm intervals over the rostral-caudal extent of the infarct. The sections were dried and stained with hematoxylin and eosin. A section from each 660-μm interval was digitized with a video camera controlled by an image analyzer. The image of each section was stored as a 1280×960 pixel matrix and displayed on a video monitor. With the observer blinded to experimental condition, the following regions of interest (ROI) were cursor outlined: non-infarcted ipsilateral cerebral cortex, non-infarcted ipsilateral subcortex, contralateral cerebral cortex, and contralateral subcortex. The area within each ROI (mm2) was determined by automated counting of calibrated pixels. Ipsilateral non-infarcted cortex and subcortex areas were subtracted from the corresponding contralateral ROI values to estimate the area of ischemic tissue damage to control for brain edema [Lin et al. Stroke 24:117-121, 1993]. Infarct volumes (mm3) were computed as running sums of subtracted infarct area multiplied by the known interval (e.g., 660 11m) between sections over the rostral-caudal extent of the infarct calculated as an orthogonal projection [Warner et al. Anesthesiology 82:1237-1245, discussion 1227A, 1995].

### Experimental Designs

The same individuals performed surgical procedures and outcome analyses in all experiments. In each experiment, rats were randomly assigned to respective treatment groups and experimenters were blind to group assignment. An a priori power analysis was conducted using data from the same model reported in prior studies [Mackensen et al. J. Neurosci. 21:4582-4592, 2001; Sheng et al. Free Radic. Biol Med. 33:947-961, 2002], which indicated that a group size of 15 rats would be sufficient to allow detection of a 40% reduction in cerebral infarct size, given 6=0.8 and P<0.05.

### Statistical Analysis

Parametric data (physiologic values, cerebral infarct volumes, and NF-κB optical densities, aconitase activities) were compared by I-way ANOVA and Fischer's protected least squares difference test when appropriate. Parametric data are expressed as mean±standard deviation. Neurologic scores were compared by the Kruskal-Wallis H statistic or Mann-Whitney U statistic where appropriate and are expressed as median±interquartile range.

### Example 1

### Effects of MnTnHex-2-PyP⁵⁺ in Murine Subarachnoid Hemorrhage

Male mice (body weight=20-25 gm) were anesthetized with isoflurane and subjected to endovascular perforation of the right anterior cerebral artery just distal to the middle cerebral artery bifurcation. Mice were allowed to recover from anesthesia and randomly assigned to treatment (225 mg/kg MnTnHex-2-PyP⁵⁺ twice per day, i.p. with treatment begun 60 min post-SAH, n=15) and vehicle (saline 0.1 ml twice a day, n=15) groups.

Seventy-two hrs post-SAH, mice were neurologically evaluated as described above, with the experimenter blinded to group assignment. Normal neurologic function was scored as 0 with the maximal deficit score=48.

The mice were anesthetized and subjected to intraluminal arterial casting for later determination of arterial cross-sectional diameter. Subarachnoid clot size was graded using a standardized scoring system.

One mouse in the MnTnHex-2-PyP⁵⁺ group died at 2 days post-SAH. Three mice died in the vehicle group (2 died 3 days post-SAH, 1 died 2 days post-SAH). Neurologic scores in surviving mice and clot size were compared with the Mann-Whitney U statistic. Vessel diameters were compared with the Student's t test.

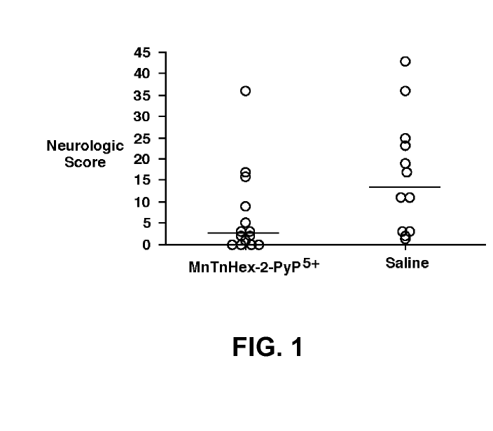

At 72 hrs post-SAH, median±interquartile range neuroscore was better (P=0.02) in mice treated with MnTnHex-2-PyP⁵⁺ (n=14, 2.5±9) than vehicle (n=12, 14±21). See FIG. 1: Open circles indicate individual mouse values. Horizontal lines indicate group median values. A score of 0=no deficit.

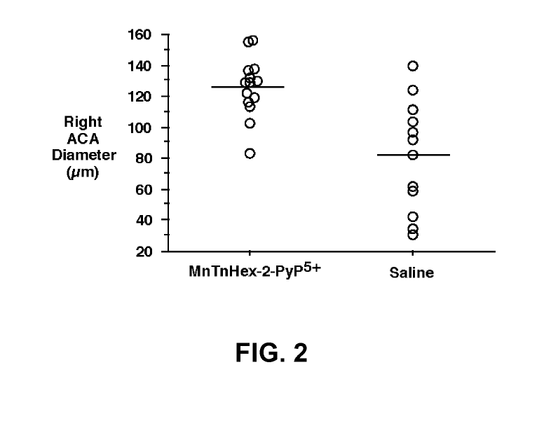

MnTnHex-2-PyP⁵⁺ increased mean±SD diameters in the right anterior cerebral artery (130±19 μm vs. 82±36 μm, P=0.0005), right middle cerebral artery (123±29 μm vs. 83±33 μm, P=0.0033), and right internal carotid artery (143±30 μm vs. 109±35 μm, P=0.015). There was no effect of treatment on basilar artery diameter (200±17 μm vs. 198±19 μm, P=0.723), consistent with lack of clot at this location. See FIG. 2: Open circles indicate individual mouse values. Horizontal lines indicate group median values.

Systemic treatment with MnTnHex-2-PyP⁵⁺, begun at a clinically relevant post-ictal interval, improved outcome from SAH defined as improvement in neurologic function. This was associated with improved vessel diameter in the vicinity of the hemorrhage.

### Example 2

### Neurologic Function After Twice Daily Injections of MnTnHex-2-PyP⁵⁺

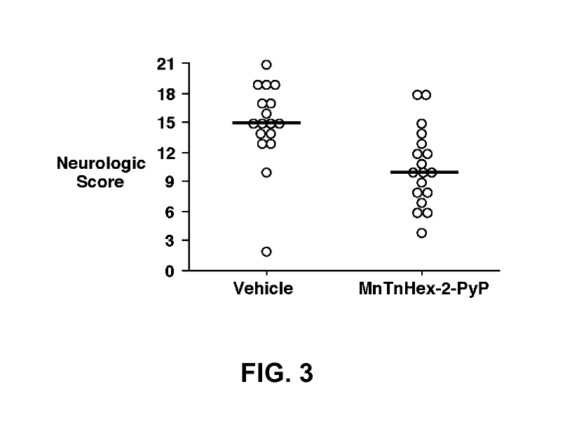

Rats were subjected to 90 min middle cerebral artery occlusion. Five minutes after reperfusion onset, they were treated with vehicle or 225 μg/kg MnTnHex-2-PyP⁵⁺ intravenously. The doses were repeatedly twice daily as subcutaneous injections for 7 days after which neurologic function was assessed as described above. See FIG. 3: Open circles indicate individual animal values. Horizontal lines indicate group median values. 0=no neurologic deficit. Neurologic score was improved in the MnTnHex-2-PyP⁵⁺ treatment group (P=0.002).

### Example 3

### Infarct Volumes After Twice Daily Injections of MnTnHex-2-PyP⁵⁺

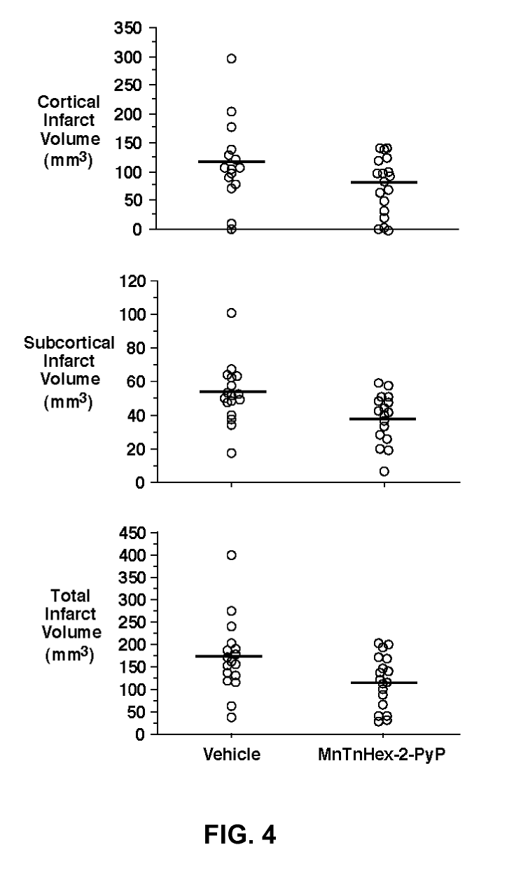

Infarct volumes measured 7 days after 90 min middle cerebral artery occlusion. Rats were treated with intravenous vehicle (0.3 ml phosphate buffered saline) or MnTnHex-2-PyP⁵⁺ (225 μg/kg) 5 min after reperfusion onset. Ten hours later twice a day subcutaneous of vehicle (0.3 ml) or MnTnHex-2-PyP⁵⁺ (225 μg/kg) were begun. Infarct volumes were measured as described above. MnTnHex-2-PyP⁵⁺ reduced cerebral infarct volume in the cortex (P=0.05), subcortex (P=0.01), which was reflected in a 32% reduction in total infarct volume (P=0.028). See FIG. 4: Open circles indicate individual animal values. Horizontal lines indicate group mean values.

### Example 4

### Neurologic Function After Twice Daily Injections of MnTnHex-2-PyP⁵⁺

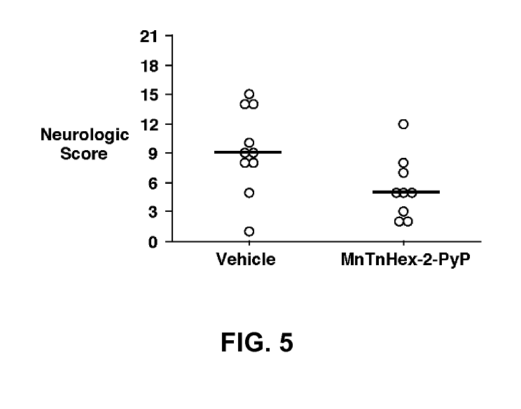

Five minutes post-treatment. Rats were subjected to 90 min MCAO. Six hours after reperfusion onset, they were treated with intra-arterial 0.3 ml phosphate buffered saline (vehicle) or 225 μg/kg MnTnHex-2-PyP⁵⁺. The same doses were given subcutaneously at the same time and continued twice daily as subcutaneous injections for 7 days after which neurologic function was assessed as described in Example 6. See FIG. 5: Open circles indicate individual animal values. Horizontal lines indicate group median values. 0=no neurologic deficit. Neurologic score was improved in the MnTnHex-2-PyP⁵⁺ treatment group (P=0.04).

### Example 5

### Infarct Volumes After Twice Daily Injections of MnTnHex-2-PyP⁵⁺

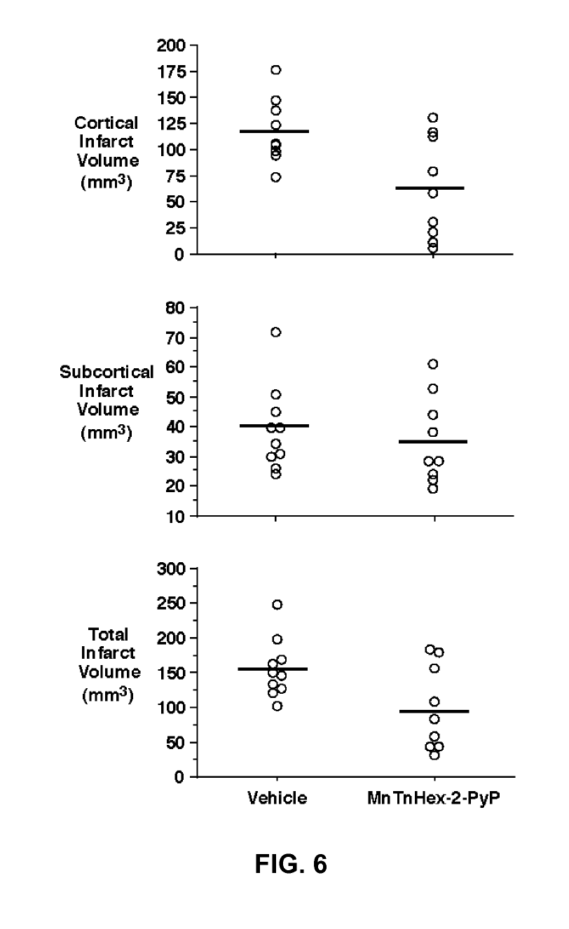

Six hours post-treatment. Cerebral infarct volumes measured 7 days after 90 min MCAO. Rats were treated with intra-arterial vehicle (0.3 ml phosphate buffered saline) or MnTnHex-2-PyP⁵⁺ (225 μg/kg) 6 hrs after reperfusion onset. The same doses were given subcutaneously at the same time and continued twice daily as subcutaneous injections for 7 days after which MnTnHex-2-PyP⁵⁺ reduced cerebral infarct volume in the cortex (P=0.01) which was reflected in a 37% reduction in total infarct volume (P=0.03). Infarct size was not changed in the subcortex (P=0.58). See FIG. 6: Open circles indicate individual animal values. Horizontal lines indicate group mean values.

### Example 6

### NF-κB Binding

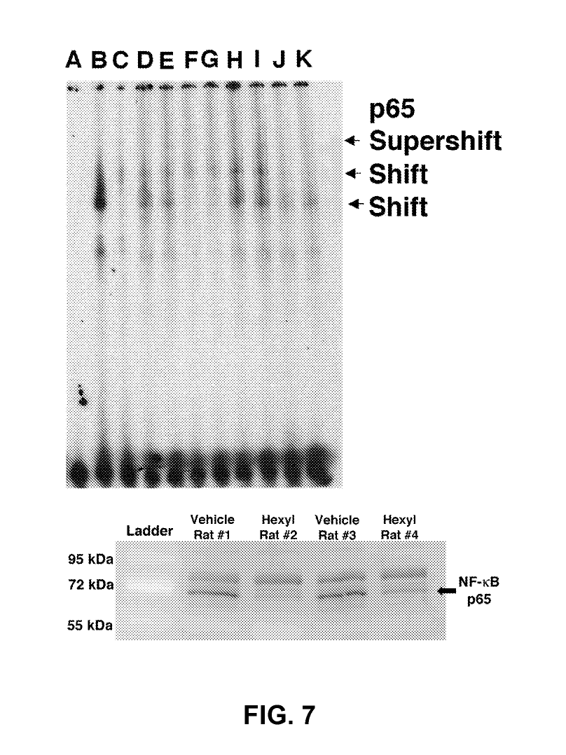

Intravenous MnTnHex-2-PyP⁵⁺ (hexyl) decreases post-ischemic NF-κB DNA binding to a κB consensus oligo due inhibition of NF-κB p65 nuclear translocation. Data are from 4 rats subjected to 90 min MCAO and then treated with vehicle or MnTnHex-2-PyP⁵⁺ (225 μg/kg IV). Six hr later, ischemic brain was harvested for EMSA performed on nuclear extracts (2.5 mg). See FIG. 7. Upper gel (EMSA): D and E (without and with p65 antibody, respectively) are rat #1 (vehicle). F and G (without and with p65 antibody, respectively) are rat #2 (hexyl). H and I=vehicle rat #3 (with and without p65). J and K=rat #4 (hexyl) with and without p65. A-C are control lanes (A=probe only, B=positive control (HeLa nuclear extract), C=cold competitor). Two slower migrating DNA binding complexes are observed (shift). The proteins in the slower migrating complexes were identified by super shift analysis with 1 mg of p65-specific antibody. Marked reduction in NF-κB binding is seen in rats #2 and #4 (both hexyl). Lower gel: 10 mg) from the same nuclear samples were immunoblotted with NF-κB p65-specific antibody, confirming NF-κB inhibition by MnTnHex-2-PyP⁵⁺.

### Example 7

### TNF-α and IL-6 Measurements

Rats were subjected to 90 min middle cerebral artery occlusion. Five min after onset of reperfusion, rats were randomly treated with vehicle (n=3) or 225 μg/kg IV MnTnHex-2-PyP⁵⁺ (n=3) followed by subcutaneous vehicle or 225 μg/kg MnTnHex-2-PyP⁵⁺, respectively, at 12 and 18 hrs post-MCAO. Brains were harvested at 24 hrs post-MCAO and analyzed for TNF-α and IL-6 by fluorescent enzyme-linked immunosorbent assay. Whole cell lysates from the whole brain tissue were obtained at the end of each experiment according to manufacture's protocol (Roche) with light modification. In brief, about 100 mg of brain tissue, which was diced into small pieces using a clean razor blade on ice, was placed into a pre-chilled microcentrifuge tube and further processed with 300 ml of ice-cold lysis buffer (50 mM Tris-HCl, pH 7.5, 150 mM NaCl, 1% Nonidet P40, 0.5% sodium deoxycholat, 0.1% SDS, protease inhibitors). Samples were homogenized 10 seconds and incubated for 30 minutes on ice. Homogenates were centrifuged at 14,000 g for 10 minutes at 4° C. Supernatants were collected and proteins were measured by a BCA Protein Assay Kit (Thermo Scientific). Cerebral levels of TNF-α and IL-6 were determined by rat specific ELISA kits (Thermo Scientific, IL) and normalized by the total amount of proteins (pg/mg).

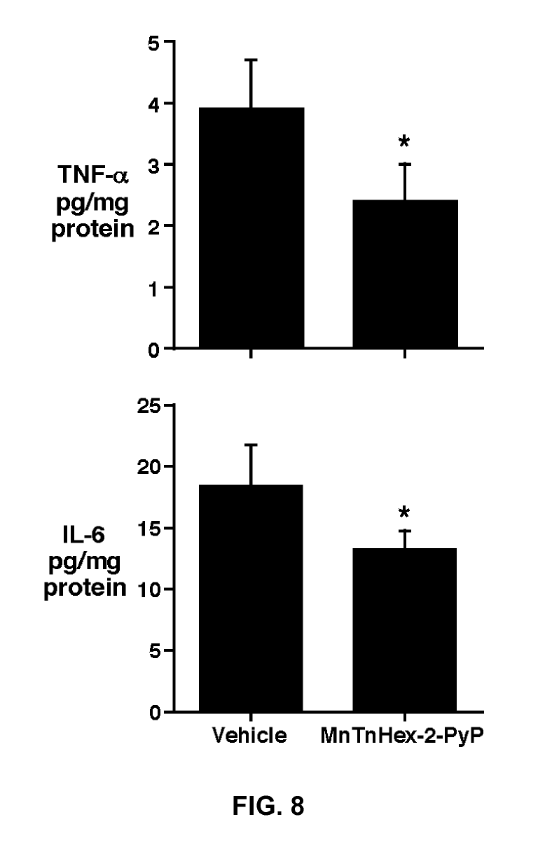

Results are shown in FIG. 8. Values represent mean±s.d. Both TNF-α and IL-6 concentrations were decreased by MnTnHex-2-PyP⁵⁺ (“P=0.04).

### Example 8

### Other Porphyrins

The methods used in Examples 1-7 may also be carried out using other substituted porphyrin compounds. For example, the substituted porphyrin may be:
Mn(III) 5,10,15,20-tetrakis(N-n-octylpyridinium-2-yl)porphyrin
Mn(III) 5,10,15,20-tetrakis(N-n-nonylpyridinium-2-yl)porphyrin
Mn(III) 5,10,15,20-tetrakis(N-n-dodecylpyridinium-2-yl)porphyrin
Mn(III) 5,10,15,20-tetrakis(N-6-methoxy-n-hexylpyridinium-2-yl)porphyrin
Mn(III) 5,10,15,20-tetrakis(N-8-methoxy-n-octylpyridinium-2-yl)porphyrin
Mn(III) 5,10,15,20-tetrakis(N-9-methoxy-n-nonylpyridinium-2-yl)porphyrin
Mn(III) 5,10,15,20-tetrakis(N-12-methoxy-n-dodecylpyridinium-2-yl)porphyrin
Mn(III) 5,10,15,20-tetrakis[N,N′-di-n-hexylimidazolium-2-yl]porphyrin
Mn(III) 5,10,15,20-tetrakis(N,N′-di-n-hexylpyrazolium-4-yl)porphyrin
Mn(III) 5,10,15,20-tetrakis(N-n-hexylthiazolium-4-yl)porphyrin
Mn(III) 5,10,15,20-tetrakis[N,N′-di-n-hexylpyridazolium-2-yl]porphyrin

1. A method of treating ischemic injury comprising administering a therapeutically effective amount of a compound of formula (I): 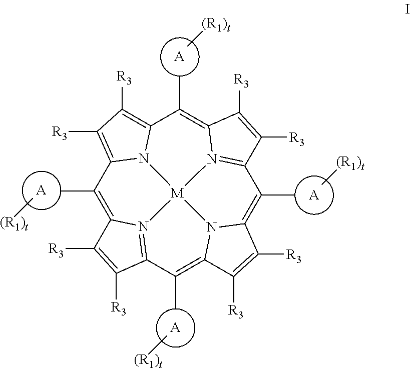
wherein:
each A is independently a heteroaryl group;
each R₁ is independently selected from H, C₆₋₁₂ alkyl, —(CH₂)ₙOR₂, —(CH₂)ₙSR₂, —(CH₂)ₙNR₂R₂, —(CH₂)ₙC(O)OR₄ and —(CH₂)ₘCHₚX_{q};
each R₂ is independently selected from hydrogen, alkyl, haloalkyl and —C(O)R₄;
each R₃ is independently selected from hydrogen, halogen, hydroxyl, alkoxy, alkyl, alkenyl, alkynyl, cycloalkyl, heterocycloalkyl, amino, amide, nitro, carboxylic acid, carboxyl, aryl, heteroaryl, thiol, thioalkyl, thioester, disulfide, phosphine, carbonyl, carbonylamino, formyl, sulfonyl, sulfonylamino, cyano, isocyano, C₁₋₄ alkyl aryl and C₁₋₄ alkyl heteroaryl;
each R₄ is independently selected from hydrogen, halogen, hydroxyl, alkoxy, alkyl, alkenyl, alkynyl, cycloalkyl, heterocycloalkyl, amino, amide, nitro, carboxylic acid, carboxyl, aryl, heteroaryl, thiol, thioalkyl, thioester, disulfide, phosphine, carbonyl, carbonylamino, formyl, sulfonyl, sulfonylamino, cyano, isocyano, C₁₋₄ alkyl aryl and C₁₋₄ alkyl heteroaryl;
each X is independently a halogen;
n is 1 to 12;
m is 1 to 11;
p is 0 to 3;
q is 0 to 3;
t is 0 to 2;
wherein p+q is 3; and
M is selected from Mn, Fe, Co, Ni, Cu, V or 2 hydrogens;
wherein when said compound bears a charge, the compound further comprises one or more counterions;
to a subject in need thereof more than 4.5 hours post ischemia onset.

2. A method of treating ischemic injury comprising administering a therapeutically effective amount of a compound of formula (I): 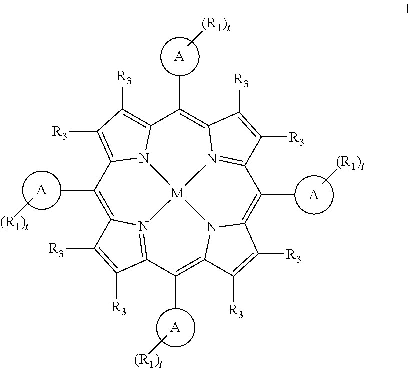
wherein:
each A is independently a heteroaryl group;
each R₁ is independently selected from H, C₆₋₁₂ alkyl, —(CH₂)ₙOR₂, —(CH₂)ₙSR₂, —(CH₂)ₙNR₂R₂, —(CH₂)ₙC(O)OR₄ and —(CH₂)ₘCHₚX_{q};
each R₂ is independently selected from hydrogen, alkyl, haloalkyl and —C(O)R₄;
each R₃ is independently selected from hydrogen, halogen, hydroxyl, alkoxy, alkyl, alkenyl, alkynyl, cycloalkyl, heterocycloalkyl, amino, amide, nitro, carboxylic acid, carboxyl, aryl, heteroaryl, thiol, thioalkyl, thioester, disulfide, phosphine, carbonyl, carbonylamino, formyl, sulfonyl, sulfonylamino, cyano, isocyano, C₁₋₄ alkyl aryl and C₁₋₄ alkyl heteroaryl;
each R₄ is independently selected from hydrogen, halogen, hydroxyl, alkoxy, alkyl, alkenyl, alkynyl, cycloalkyl, heterocycloalkyl, amino, amide, nitro, carboxylic acid, carboxyl, aryl, heteroaryl, thiol, thioalkyl, thioester, disulfide, phosphine, carbonyl, carbonylamino, formyl, sulfonyl, sulfonylamino, cyano, isocyano, C₁₋₄ alkyl aryl and C₁₋₄ alkyl heteroaryl;
each X is independently a halogen;
n is 1 to 12;
m is 1 to 11;
p is 0 to 3;
q is 0 to 3;
t is 0 to 2;
wherein p+q is 3; and
M is selected from Mn, Fe, Co, Ni, Cu, V or 2 hydrogens;
wherein when said compound bears a charge, the compound further comprises one or more counterions;
to a subject in need thereof more than 6 hours post ischemia onset.

3. A method of treating ischemic injury comprising administering a therapeutically effective amount of a compound of formula (I): 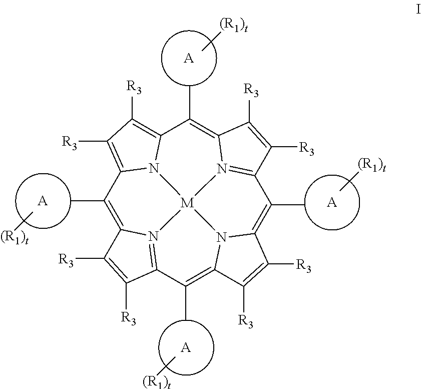
wherein:
each A is independently a heteroaryl group;
each R₁ is independently selected from H, C₆₋₁₂ alkyl, —(CH₂)ₙOR₂, —(CH₂)ₙSR₂, —(CH₂)ₙNR₂R₂, —(CH₂)ₙC(O)OR₄ and —(CH₂)ₘCHₚX_{q};
each R₂ is independently selected from hydrogen, alkyl, haloalkyl and —C(O)R₄;
each R₃ is independently selected from hydrogen, halogen, hydroxyl, alkoxy, alkyl, alkenyl, alkynyl, cycloalkyl, heterocycloalkyl, amino, amide, nitro, carboxylic acid, carboxyl, aryl, heteroaryl, thiol, thioalkyl, thioester, disulfide, phosphine, carbonyl, carbonylamino, formyl, sulfonyl, sulfonylamino, cyano, isocyano, C₁₋₄ alkyl aryl and C₁₋₄ alkyl heteroaryl;
each R₄ is independently selected from hydrogen, halogen, hydroxyl, alkoxy, alkyl, alkenyl, alkynyl, cycloalkyl, heterocycloalkyl, amino, amide, nitro, carboxylic acid, carboxyl, aryl, heteroaryl, thiol, thioalkyl, thioester, disulfide, phosphine, carbonyl, carbonylamino, formyl, sulfonyl, sulfonylamino, cyano, isocyano, C₁₋₄ alkyl aryl and C₁₋₄ alkyl heteroaryl;
each X is independently a halogen;
n is 1 to 12;
m is 1 to 11;
p is 0 to 3;
q is 0 to 3;
t is 0 to 2;
wherein p+q is 3; and
M is selected from Mn, Fe, Co, Ni, Cu, V or 2 hydrogens;
wherein when said compound bears a charge, the compound further comprises one or more counterions;
to a subject in need thereof at least once per day for at least 5 days post ischemia onset.

4. A method of providing neuroprotection comprising administering a therapeutically effective amount of a compound of formula (I): 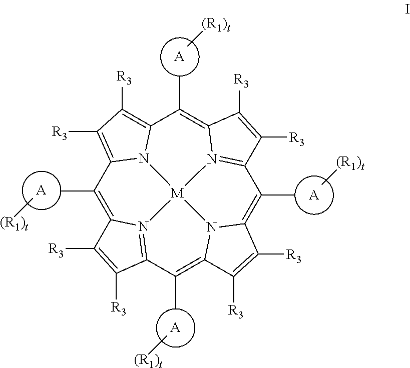
wherein:
each A is independently a heteroaryl group;
each R₁ is independently selected from H, C₆₋₁₂ alkyl, —(CH₂)ₙOR₂, —(CH₂)ₙSR₂, —(CH₂)ₙNR₂R₂, —(CH₂)ₙC(O)OR₄ and —(CH₂)ₘCHₚX_{q};
each R₂ is independently selected from hydrogen, alkyl, haloalkyl and —C(O)R₄;
each R₃ is independently selected from hydrogen, halogen, hydroxyl, alkoxy, alkyl, alkenyl, alkynyl, cycloalkyl, heterocycloalkyl, amino, amide, nitro, carboxylic acid, carboxyl, aryl, heteroaryl, thiol, thioalkyl, thioester, disulfide, phosphine, carbonyl, carbonylamino, formyl, sulfonyl, sulfonylamino, cyano, isocyano, C₁₋₄ alkyl aryl and C₁₋₄ alkyl heteroaryl;
each R₄ is independently selected from hydrogen, halogen, hydroxyl, alkoxy, alkyl, alkenyl, alkynyl, cycloalkyl, heterocycloalkyl, amino, amide, nitro, carboxylic acid, carboxyl, aryl, heteroaryl, thiol, thioalkyl, thioester, disulfide, phosphine, carbonyl, carbonylamino, formyl, sulfonyl, sulfonylamino, cyano, isocyano, C₁₋₄ alkyl aryl and C₁₋₄ alkyl heteroaryl;
each X is independently a halogen;
n is 1 to 12;
m is 1 to 11;
p is 0 to 3;
q is 0 to 3;
t is 0 to 2;
wherein p+q is 3; and
M is selected from Mn, Fe, Co, Ni, Cu, V or 2 hydrogens;
wherein when said compound bears a charge, the compound further comprises one or more counterions;
to a subject in need thereof more than 4.5 hours post ischemia onset.

5. A method of providing neuroprotection comprising administering a therapeutically effective amount of a compound of formula (I): 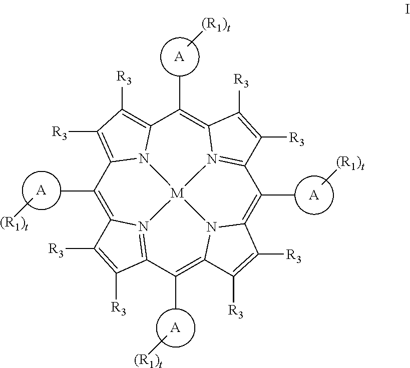
wherein:
each A is independently a heteroaryl group;
each R₁ is independently selected from H, C₆₋₁₂ alkyl, —(CH₂)ₙOR₂, —(CH₂)ₙSR₂, —(CH₂)ₙNR₂R₂, —(CH₂)ₙC(O)OR₄ and —(CH₂)ₘCHₚX_{q};
each R₂ is independently selected from hydrogen, alkyl, haloalkyl and —C(O)R₄;
each R₃ is independently selected from hydrogen, halogen, hydroxyl, alkoxy, alkyl, alkenyl, alkynyl, cycloalkyl, heterocycloalkyl, amino, amide, nitro, carboxylic acid, carboxyl, aryl, heteroaryl, thiol, thioalkyl, thioester, disulfide, phosphine, carbonyl, carbonylamino, formyl, sulfonyl, sulfonylamino, cyano, isocyano, C₁₋₄ alkyl aryl and C₁₋₄ alkyl heteroaryl;
each R₄ is independently selected from hydrogen, halogen, hydroxyl, alkoxy, alkyl, alkenyl, alkynyl, cycloalkyl, heterocycloalkyl, amino, amide, nitro, carboxylic acid, carboxyl, aryl, heteroaryl, thiol, thioalkyl, thioester, disulfide, phosphine, carbonyl, carbonylamino, formyl, sulfonyl, sulfonylamino, cyano, isocyano, C₁₋₄ alkyl aryl and C₁₋₄ alkyl heteroaryl;
each X is independently a halogen;
n is 1 to 12;
m is 1 to 11;
p is 0 to 3;
q is 0 to 3;
t is 0 to 2;
wherein p+q is 3; and
M is selected from Mn, Fe, Co, Ni, Cu, V or 2 hydrogens;
wherein when said compound bears a charge, the compound further comprises one or more counterions;
to a subject in need thereof more than 6 hours post ischemia onset.

6. A method of providing neuroprotection comprising administering a therapeutically effective amount of a compound of formula (I): 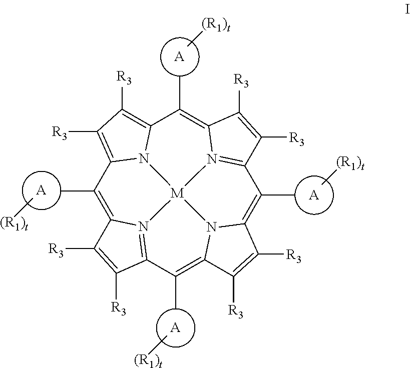
wherein:
each A is independently a heteroaryl group;
each R₁ is independently selected from H, C₆₋₁₂ alkyl, —(CH₂)ₙOR₂, —(CH₂)ₙSR₂, —(CH₂)ₙNR₂R₂, —(CH₂)ₙC(O)OR₄ and —(CH₂)ₘCHₚX_{q};
each R₂ is independently selected from hydrogen, alkyl, haloalkyl and —C(O)R₄;
each R₃ is independently selected from hydrogen, halogen, hydroxyl, alkoxy, alkyl, alkenyl, alkynyl, cycloalkyl, heterocycloalkyl, amino, amide, nitro, carboxylic acid, carboxyl, aryl, heteroaryl, thiol, thioalkyl, thioester, disulfide, phosphine, carbonyl, carbonylamino, formyl, sulfonyl, sulfonylamino, cyano, isocyano, C₁₋₄ alkyl aryl and C₁₋₄ alkyl heteroaryl;
each R₄ is independently selected from hydrogen, halogen, hydroxyl, alkoxy, alkyl, alkenyl, alkynyl, cycloalkyl, heterocycloalkyl, amino, amide, nitro, carboxylic acid, carboxyl, aryl, heteroaryl, thiol, thioalkyl, thioester, disulfide, phosphine, carbonyl, carbonylamino, formyl, sulfonyl, sulfonylamino, cyano, isocyano, C₁₋₄ alkyl aryl and C₁₋₄ alkyl heteroaryl;
each X is independently a halogen;
n is 1 to 12;
m is 1 to 11;
p is 0 to 3;
q is 0 to 3;
t is 0 to 2;
wherein p+q is 3; and
M is selected from Mn, Fe, Co, Ni, Cu, V or 2 hydrogens;
wherein when said compound bears a charge, the compound further comprises one or more counterions;
to a subject in need thereof at least once per day for at least 5 days post ischemia onset.

7. The method of any one of claims 1-3, wherein the ischemic injury is selected from cerebral ischemia, stroke, spinal cord injury and traumatic brain injury.

8. The method of any one of claims 1-7, wherein the substituted porphyrin is administered more than about 6 hours post ischemia onset.

9. The method of any one of claims 1-8, wherein the substituted porphyrin is administered more than about 8 hours post ischemia onset.

10. The method of any one of claims 1-9, wherein the substituted porphyrin is administered more than about 10 hours post ischemia onset.

11. The method of any one of claims 1-10, wherein the substituted porphyrin is administered more than about 4.5 hours post reperfusion.

12. The method of any one of claims 1-11, wherein the substituted porphyrin is administered for about 1 week post ischemia onset.

13. The method of any one of claims 1-12, wherein the substituted porphyrin is administered for about 2 weeks post ischemia onset.

14. The method of any one of claims 1-13, wherein the substituted porphyrin is administered for about 3 weeks post ischemia onset.

15. The method of any one of claims 1-14, wherein the substituted porphyrin is administered for about 4 weeks post ischemia onset.

16. The method of any one of claims 1-15, wherein the substituted porphyrin is administered once weekly.

17. The method of any one of claims 1-16, wherein the substituted porphyrin is administered twice weekly.

18. A method of treating subarachnoid hemorrhage comprising administering a therapeutically effective amount of a compound of formula (I): 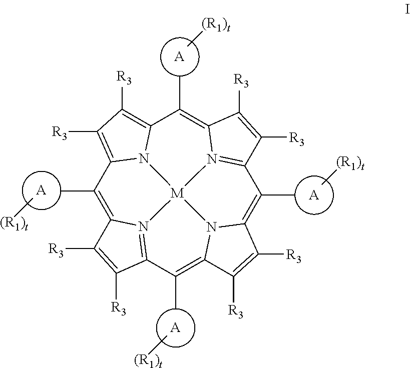
wherein:
each A is independently a heteroaryl group;
each R₁ is independently selected from H, C₆₋₁₂ alkyl, —(CH₂)ₙOR₂, —(CH₂)ₙSR₂, —(CH₂)ₙNR₂R₂, —(CH₂)ₙC(O)OR₄ and —(CH₂)ₘCHₚX_{q};
each R₂ is independently selected from hydrogen, alkyl, haloalkyl and —C(O)R₄;
each R₃ is independently selected from hydrogen, halogen, hydroxyl, alkoxy, alkyl, alkenyl, alkynyl, cycloalkyl, heterocycloalkyl, amino, amide, nitro, carboxylic acid, carboxyl, aryl, heteroaryl, thiol, thioalkyl, thioester, disulfide, phosphine, carbonyl, carbonylamino, formyl, sulfonyl, sulfonylamino, cyano, isocyano, C₁₋₄ alkyl aryl and C₁₋₄ alkyl heteroaryl;
each R₄ is independently selected from hydrogen, halogen, hydroxyl, alkoxy, alkyl, alkenyl, alkynyl, cycloalkyl, heterocycloalkyl, amino, amide, nitro, carboxylic acid, carboxyl, aryl, heteroaryl, thiol, thioalkyl, thioester, disulfide, phosphine, carbonyl, carbonylamino, formyl, sulfonyl, sulfonylamino, cyano, isocyano, C₁₋₄ alkyl aryl and C₁₋₄ alkyl heteroaryl;
each X is independently a halogen;
n is 1 to 12;
m is 1 to 11;
p is 0 to 3;
q is 0 to 3;
t is 0 to 2;
wherein p+q is 3; and
M is selected from Mn, Fe, Co, Ni, Cu, V or 2 hydrogens;
wherein when said compound bears a charge, the compound further comprises one or more counterions;
to a subject in need thereof.

19. The method of claim 18, wherein the substituted porphyrin is administered more than about 6 hours post hemorrhage.

20. The method of claim 18 or 19, wherein the substituted porphyrin is administered more than about 8 hours post hemorrhage.

21. The method of any one of claims 18-20, wherein the substituted porphyrin is administered more than about 10 hours post hemorrhage.

22. The method of any one of claims 18-21, wherein the substituted porphyrin is administered to the subject in need thereof at least once per day for at least 5 days post hemorrhage.

23. The method of any one of claims 18-22, wherein the substituted porphyrin is administered for about 1 week post hemorrhage.

24. The method of any one of claims 18-23, wherein the substituted porphyrin is administered for about 2 weeks post hemorrhage.

25. The method of any one of claims 18-24, wherein the substituted porphyrin is administered for about 3 weeks post hemorrhage.

26. The method of any one of claims 18-25, wherein the substituted porphyrin is administered for about 4 weeks post hemorrhage.

27. A method of treating traumatic brain injury (TBI) comprising administering a therapeutically effective amount of a compound of formula (I): 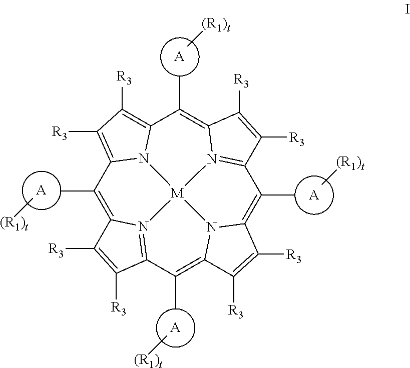
wherein:
each A is independently a heteroaryl group;
each R₁ is independently selected from H, C₆₋₁₂ alkyl, —(CH₂)ₙOR₂, —(CH₂)ₙSR₂, —(CH₂)ₙNR₂R₂, —(CH₂)ₙC(O)OR₄ and —(CH₂)ₘCHₚX_{q};
each R₂ is independently selected from hydrogen, alkyl, haloalkyl and —C(O)R₄;
each R₃ is independently selected from hydrogen, halogen, hydroxyl, alkoxy, alkyl, alkenyl, alkynyl, cycloalkyl, heterocycloalkyl, amino, amide, nitro, carboxylic acid, carboxyl, aryl, heteroaryl, thiol, thioalkyl, thioester, disulfide, phosphine, carbonyl, carbonylamino, formyl, sulfonyl, sulfonylamino, cyano, isocyano, C₁₋₄ alkyl aryl and C₁₋₄ alkyl heteroaryl;
each R₄ is independently selected from hydrogen, halogen, hydroxyl, alkoxy, alkyl, alkenyl, alkynyl, cycloalkyl, heterocycloalkyl, amino, amide, nitro, carboxylic acid, carboxyl, aryl, heteroaryl, thiol, thioalkyl, thioester, disulfide, phosphine, carbonyl, carbonylamino, formyl, sulfonyl, sulfonylamino, cyano, isocyano, C₁₋₄ alkyl aryl and C₁₋₄ alkyl heteroaryl;
each X is independently a halogen;
n is 1 to 12;
m is 1 to 11;
p is 0 to 3;
q is 0 to 3;
t is 0 to 2;
wherein p+q is 3; and
M is selected from Mn, Fe, Co, Ni, Cu, V or 2 hydrogens;
wherein when said compound bears a charge, the compound further comprises one or more counterions;
to a subject in need thereof.

28. The method of claim 27, wherein the substituted porphyrin is administered more than about 6 hours post TBI.

29. The method of claim 27 or 28, wherein the substituted porphyrin is administered more than about 8 hours post TBI.

30. The method of any one of claims 27-29, wherein the substituted porphyrin is administered more than about 10 hours post TBI.

31. The method of any one of claims 27-30, wherein the substituted porphyrin is administered to the subject in need thereof at least once per day for at least 5 days post TBI.

32. The method of any one of claims 27-31, wherein the substituted porphyrin is administered for about 1 week post TBI.

33. The method of any one of claims 27-32, wherein the substituted porphyrin is administered for about 2 weeks post TBI.

34. The method of any one of claims 27-33, wherein the substituted porphyrin is administered for about 3 weeks post TBI.

35. The method of any one of claims 27-34, wherein the substituted porphyrin is administered for about 4 weeks post TBI.

36. A method of treating spinal cord injury (SCI) comprising administering a therapeutically effective amount of a compound of formula (I): 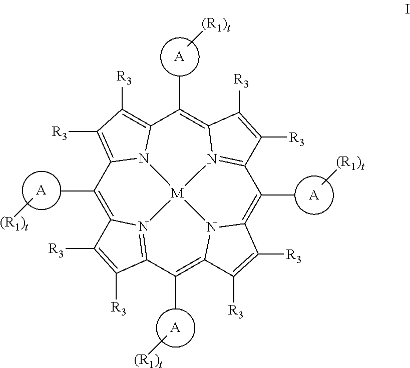
wherein:
each A is independently a heteroaryl group;
each R₁ is independently selected from H, C₆₋₁₂ alkyl, —(CH₂)ₙOR₂, —(CH₂)ₙSR₂, —(CH₂)ₙNR₂R₂, —(CH₂)ₙC(O)OR₄ and —(CH₂)ₘCHₚX_{q};
each R₂ is independently selected from hydrogen, alkyl, haloalkyl and —C(O)R₄;
each R₃ is independently selected from hydrogen, halogen, hydroxyl, alkoxy, alkyl, alkenyl, alkynyl, cycloalkyl, heterocycloalkyl, amino, amide, nitro, carboxylic acid, carboxyl, aryl, heteroaryl, thiol, thioalkyl, thioester, disulfide, phosphine, carbonyl, carbonylamino, formyl, sulfonyl, sulfonylamino, cyano, isocyano, C₁₋₄ alkyl aryl and C₁₋₄ alkyl heteroaryl;
each R₄ is independently selected from hydrogen, halogen, hydroxyl, alkoxy, alkyl, alkenyl, alkynyl, cycloalkyl, heterocycloalkyl, amino, amide, nitro, carboxylic acid, carboxyl, aryl, heteroaryl, thiol, thioalkyl, thioester, disulfide, phosphine, carbonyl, carbonylamino, formyl, sulfonyl, sulfonylamino, cyano, isocyano, C₁₋₄ alkyl aryl and C₁₋₄ alkyl heteroaryl;
each X is independently a halogen;
n is 1 to 12;
m is 1 to 11;
p is 0 to 3;
q is 0 to 3;
t is 0 to 2;
wherein p+q is 3; and
M is selected from Mn, Fe, Co, Ni, Cu, V or 2 hydrogens;
wherein when said compound bears a charge, the compound further comprises one or more counterions;
to a subject in need thereof.

37. The method of claim 36, wherein the substituted porphyrin is administered more than about 6 hours post SCI.

38. The method of claim 36 or 37, wherein the substituted porphyrin is administered more than about 8 hours post SCI.

39. The method of any one of claims 36-38, wherein the substituted porphyrin is administered more than about 10 hours post SCI.

40. The method of any one of claims 36-39, wherein the substituted porphyrin is administered to the subject in need thereof at least once per day for at least 5 days post SCI.

41. The method of any one of claims 36-40, wherein the substituted porphyrin is administered for about 1 week post SCI.

42. The method of any one of claims 36-41, wherein the substituted porphyrin is administered for about 2 weeks post SCI.

43. The method of any one of claims 36-42, wherein the substituted porphyrin is administered for about 3 weeks post SCI.

44. The method of any one of claims 36-43, wherein the substituted porphyrin is administered for about 4 weeks post SCI.

45. The method of any one of the preceding claims, wherein in the compound of formula (I):
each A is independently a pyridyl group;
each R₁ is independently H or C₆₋₁₂ alkyl;
each R₃ is independently selected from hydrogen, halogen, hydroxyl, alkoxy, alkyl, alkenyl, alkynyl, cycloalkyl, heterocycloalkyl, amino, amide, nitro, carboxylic acid, carboxyl, aryl, heteroaryl, thiol, thioalkyl, thioester, disulfide, phosphine, carbonyl, carbonylamino, formyl, sulfonyl, sulfonylamino, cyano, isocyano, C₁₋₄ alkyl aryl and C₁₋₄ alkyl heteroaryl; and
M is selected from Mn, Fe, Co, Ni, Cu, V or 2 hydrogens.

46. The method of any one of the preceding claims, wherein the substituted porphyrin is administered once per day.

47. The method of any one of the preceding claims, wherein the substituted porphyrin is administered twice per day.

48. The method of any one of the preceding claims, wherein the substituted porphyrin is administered three times per day.

49. The method of any one of the preceding claims, wherein the substituted porphyrin is administered four times per day.

50. The method of any one of the preceding claims, wherein the substituted porphyrin is administered continuously.

51. The method of any one of the preceding claims, wherein the substituted porphyrin is administered via intravenous administration.

52. The method of any one of the preceding claims, wherein M is Mn.

53. The method of any one of the preceding claims, wherein each R₁ is independently selected from the group consisting of —(CH₂)₅CH₃, —(CH₂)₈CH₃, —(CH₂)₂OCH₃, —(CH₂)₆OCH₃, —(CH₂)₆OCH₂CH₃, —(CH₂)₆OCH(CH₃)₂, —(CH₂)₆OC(CH₃)₃, —(CH₂)₆OCF₃, —(CH₂)₆OCH₂CF₃, —(CH₂)₆OH, —(CH₂)₂SCH₃, —(CH₂)₆SCH₃, —(CH₂)₆NH₂, —(CH₂)₅CH₂F, —(CH₂)₅CHF₂, or —(CH₂)₅CF₃.

54. The method of any one of the preceding claims, wherein each R₁ is independently a C₆₋₁₂ alkyl group.

55. The method of any one of the preceding claims, wherein each R₁ is n-hexyl.

56. The method of any one of the preceding claims, wherein each R₁ is n-octyl.

57. The method of any one of the preceding claims, wherein each R₁ is n-nonyl.

58. The method of any one of the preceding claims, wherein each R₁ is n-dodecyl.

59. The method of any one of the preceding claims, wherein each R₁ is a substituted C₆₋₁₂ alkyl group.

60. The method of any one of the preceding claims, wherein each A is independently a pyridyl group.

61. The method of any one of the preceding claims, wherein each A is a 2-pyridyl group.

62. The method of any one of the preceding claims, wherein each A is a 3-pyridyl group.

63. The method of any one of the preceding claims, wherein each A is a 4-pyridyl group.

64. The method of any one of the preceding claims, wherein each A is an imidazolyl group.

65. The method of any one of the preceding claims, wherein each A is a thiazolyl group.

66. The method of any one of the preceding claims, wherein each A is a pyrazolyl group.

67. The method of any one of the preceding claims, wherein each A is a pyrimidyl group.
